SEQUENCE LISTING (16 sequences)

SEQ ID NO: 1 - DNA, 3393 bp, Homo Sapiens (Wild-type BCR-ABL)
  CDS (1)..(3393)
atgttggagatctgcctgaagctggtgggctgcaaatccaagaaggggctgtcctcgtcctccagctgttatctggaagaagcccttcagcggccagtagcatctgactttgagcctcagggtctgagtgaagccgctcgttggaactccaaggaaaaccttctcgctggacccagtgaaaatgaccccaaccttttcgttgcactgtatgattttgtggccagtggagataacactctaagcataactaaaggtgaaaagctccgggtcttaggctataatcacaatggggaatggtgtgaagcccaaaccaaaaatggccaaggctgggtcccaagcaactacatcacgccagtcaacagtctggagaaacactcctggtaccatgggcctgtgtcccgcaatgccgctgagtatccgctgagcagcgggatcaatggcagcttcttggtgcgtgagagtgagagcagtcctagccagaggtccatctcgctgagatacgaagggagggtgtaccattacaggatcaacactgcttctgatggcaagctctacgtctcctccgagagccgcttcaacaccctggccgagttggttcatcatcattcaacggtggccgacgggctcatcaccacgctccattatccagccccaaagcgcaacaagcccactgtctatggtgtgtcccccaactacgacaagtgggagatggaacgcacggacatcaccatgaagcacaagctgggcgggggccagtacggggaggtgtacgagggcgtgtggaagaaatacagcctgacggtggccgtgaagaccttgaaggaggacaccatggaggtggaagagttcttgaaagaagctgcagtcatgaaagagatcaaacaccctaacctagtgcagctccttggggtctgcacccgggagcccccgttctatatcatcactgagttcatgacctacgggaacctcctggactacctgagggagtgcaaccggcaggaggtgaacgccgtggtgctgctgtacatggccactcagatctcgtcagccatggagtacctagagaagaaaaacttcatccacagagatcttgctgcccgaaactgcctggtaggggagaaccacttggtgaaggtagctgattttggcctgagcaggttgatgacaggggacacctacacagcccatgctggagccaagttccccatcaaatggactgcacccgagagcctggcctacaacaagttctccatcaagtccgacgtctgggcatttggagtattgctttgggaaattgctacctatggcatgtccccttacccgggaattgaccgttcccaggtgtatgagctgctagagaaggactaccgcatgaagcgcccagaaggctgcccagagaaggtctatgaactcatgcgagcatgttggcagtggaatccctctgaccggccctcctttgctgaaatccaccaagcctttgaaacaatgttccaggaatccagtatctcagacgaagtggaaaaggagctggggaaacaaggcgtccgtggggctgtgactaccttgctgcaggccccagagctgcccaccaagacgaggacctccaggagagctgcagagcacagagacaccactgacgtgcctgagatgcctcactccaagggccagggagagagcgatcctctggaccatgagcctgccgtgtctccattgctccctcgaaaagagcgaggtcccccggagggcggcctgaatgaagatgagcgccttctccccaaagacaaaaagaccaacttgttcagcgccttgatcaagaagaagaagaagacagccccaacccctcccaaacgcagcagctccttccgggagatggacggccagccggagcgcagaggggccggcgaggaagagggccgagacatcagcaacggggcactggctttcacccccttggacacagctgacccagccaagtccccaaagcccagcaatggggctggggtccccaatggagccctccgggagtccgggggctcaggcttccggtctccccacctgtggaagaagtccagcacgctgaccagcagccgcctagccaccggcgaggaggagggcggtggcagctccagcaagcgcttcctgcgctcttgctccgtctcctgcgttccccatggggccaaggacacggagtggaggtcagtcacgctgcctcgggacttgcagtccacgggaagacagtttgactcgtccacatttggagggcacaaaagtgagaagccggctctgcctcggaagagggcaggggagaacaggtctgaccaggtgacccgaggcacagtaacgcctccccccaggctggtgaaaaagaatgaggaagctgctgatgaggtcttcaaagacatcatggagtccagcccgggctccagcccgcccaacctgactccaaaacccctccggcggcaggtcaccgtggcccctgcctcgggcctcccccacaaggaagaagcctggaaaggcagtgccttagggacccctgctgcagctgagccagtgacccccaccagcaaagcaggctcaggtgcaccaaggggcaccagcaagggccccgccgaggagtccagagtgaggaggcacaagcactcctctgagtcgccagggagggacaaggggaaattgtccaagctcaaacctgccccgccgcccccaccagcagcctctgcagggaaggctggaggaaagccctcgcagaggcccggccaggaggctgccggggaggcagtcttgggcgcaaagacaaaagccacgagtctggttgatgctgtgaacagtgacgctgccaagcccagccagccggcagagggcctcaaaaagcccgtgctcccggccactccaaagccacaccccgccaagccgtcggggacccccatcagcccagcccccgttcccctttccacgttgccatcagcatcctcggccttggcaggggaccagccgtcttccactgccttcatccctctcatatcaacccgagtgtctcttcggaaaacccgccagcctccagagcgggccagcggcgccatcaccaagggcgtggtcttggacagcaccgaggcgctgtgcctcgccatctctgggaactccgagcagatggccagccacagcgcagtgctggaggccggcaaaaacctctacacgttctgcgtgagctatgtggattccatccagcaaatgaggaacaagtttgccttccgagaggccatcaacaaactggagaataatctccgggagcttcagatctgcccggcgtcagcaggcagtggtccggcggccactcaggacttcagcaagctcctcagttcggtgaaggaaatcagtgacatagtgcagaggtag

SEQ ID NO: 2 - protein, 1130 aa, Homo Sapiens (Wild-type BCR-ABL)
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYPLSSGINGSFLVRESESSPSQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDRSQVYELLEKDYRMKRPEGCPEKVYELMRACWQWNPSDRPSFAEIHQAFETMFQESSISDEVEKELGKQGVRGAVTTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSVSCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAWKGSALGTPAAAEPVTPTSKAGSGAPRGTSKGPAEESRVRRHKHSSESPGRDKGKLSKLKPAPPPPPAASAGKAGGKPSQRPGQEAAGEAVLGAKTKATSLVDAVNSDAAKPSQPAEGLKKPVLPATPKPHPAKPSGTPISPAPVPLSTLPSASSALAGDQPSSTAFIPLISTRVSLRKTRQPPERASGAITKGVVLDSTEALCLAISGNSEQMASHSAVLEAGKNLYTFCVSYVDSIQQMRNKFAFREAINKLENNLRELQICPASAGSGPAATQDFSKLLSSVKEISDIVQR

SEQ ID NO: 3 - DNA, 22 bp, Artificial Sequence
  Primer
gaagcttctccctgacatccgt

SEQ ID NO: 4 - DNA, 22 bp, Artificial Sequence
  Primer
gccaggctctcgggtgcagtcc

SEQ ID NO: 5 - DNA, 24 bp, Artificial Sequence
  Primer
gcgcaacaagcccactgtctatgg

SEQ ID NO: 6 - DNA, 24 bp, Artificial Sequence
  Primer
cgccagggttttcccagtcacgac

SEQ ID NO: 7 - DNA, 24 bp, Artificial Sequence
  Primer
agcggataacaatttcacacagga

SEQ ID NO: 8 - DNA, 20 bp, Artificial Sequence
  Primer
gcagagtcagaatccttcag

SEQ ID NO: 9 - DNA, 20 bp, Artificial Sequence
  Primer
tttgtaaaaggctgcccggc

SEQ ID NO: 10 - DNA, 30 bp, Artificial Sequence
  Primer
cggcattgcgggacacaggcccatggtacc

SEQ ID NO: 11 - DNA, 22 bp, Artificial Sequence
  Primer
tgaccaactcgtgtgtgaaact

SEQ ID NO: 12 - DNA, 34 bp, Artificial Sequence
  Primer
ggggaattcgccaccatgttggagatctgcctga

SEQ ID NO: 13 - DNA, 20 bp, Artificial Sequence
  Primer
ttcaccaccatggagaaggc

SEQ ID NO: 14 - DNA, 20 bp, Artificial Sequence
  Primer
caggaaatgagcttgacaaa

SEQ ID NO: 15 - DNA, 74 bp, Artificial Sequence
  Primer
cggccatatgcacgaccgaaaacctgtattttcaggtgccatggatccgtcctcccccaactacgacaagtggg

SEQ ID NO: 16 - DNA, 50 bp, Artificial Sequence
  Primer
ggccgaattcttattaggtgccacgtttccccagctccttttccacttcg

(19) United States
(12) Patent Application Publication
(10) Pub. No.: US 2010/0029676 A1
(43) Pub. Date: Feb. 4, 2010

(54) T315A AND F317I MUTATIONS OF BCR-ABL KINASE DOMAIN

(76) Inventors: Charles L. Sawyers, Los Angeles, CA (US); Michael Burgess, Los Angeles, CA (US); Neil Pravin Shah, Los Angeles, CA (US); Francis Y. Lee, Yardley, PA (US); John S. Tokarski, Princeton, NJ (US); Herbert E. Klei, Lawrenceville, NJ (US)

Correspondence Address: BRISTOL - MYERS SQUIBB COMPANY;PATENT DEPARTMENT, PO BOX 4000, PRINCETON, NJ, 08543-4000, US

(21) Appl. No.: 12/092,360

(22) PCT Filed: Nov. 3, 2006
(86) PCT No.: PCT/US06/43019

§ 371 Date: Sep. 18, 2008

Related U.S. Application Data
(60) Provisional application No. 60/733,385, filed on Nov. 4, 2005.

(51) Int. Cl. A61K 31/497; C12Q 1/48; G01N 33/53; C12Q 1/68

(52) U.S. Cl. 514/252.19; 435/15; 435/7.1; 435/6

(57) ABSTRACT

The present invention relates to mutant BCR-ABL kinase proteins, and to diagnostic and therapeutic methods and compositions useful in the management of disorders, for example cancers, involving cells that express such mutant BCR-ABL kinase proteins.

### CROSS-REFERENCE TO RELATED APPLICATIONS

This application claims benefit to provisional application U.S. Ser. No. 60/733,385 filed Nov. 4, 2005, incorporated herein by reference in its entirety.

### FIELD

The present invention relates to mutant BCR-ABL kinase proteins, and to diagnostic and therapeutic methods and compositions useful in the management of disorders, for example cancers, involving cells that express such mutant BCR-ABL kinase proteins.

### BACKGROUND

Cancer is the second leading cause of human death next to coronary disease. Worldwide, millions of people die from cancer every year. In the United States alone, cancer causes the death of well over a half-million people annually, with some 1.4 million new cases diagnosed per year. While deaths from heart disease have been declining significantly, those resulting from cancer generally are on the rise and are predicted to become the leading cause of death in the developed world.

Chronic Myelogenous Leukemia (CML) is a myeloproliferative disorder that is characterized by Philadelphia chromosome translocation. (see, e.g. C. L. Sawyers, N. En. J. Med. 340, 1330 (1999); and S. Faderl et al., N. Engl. J. Med. 341, 164 (1999)). A reciprocal translocation between chromosomes 9 and 22 produces the oncogenic BCR-ABL fusion protein. The BCL-ABL protein constitutes tyrosine kinase activity and is known to produce CML-like disease in mice (see, e.g. J. B. Konopka et al., Proc. Natl. Acad. Sci. U.S. 82, 1810 (1985); G. Q. Daley et al., Science 247, 824 (1990); and N. Heisterkamp et al., Nature 344, 251 (1990)). In fact, 95% of CML is Philadelphia-positive (Ph+). A single mutation on BCR-ABL

CML progresses through distinct clinical stages. The earliest stage, termed chronic phase, is characterized by expansion of terminally differentiated neutrophils. Over several years the disease progresses to an acute phase termed blast crisis, characterized by maturation arrest with excessive numbers of undifferentiated myeloid or lymphoid progenitor cells. The BCR-ABL oncogene is expressed at all stages, but blast crisis is characterized by multiple additional genetic and molecular changes.

Imatinib mesylate (also known as STI-571) is a potent BCR-ABL tyrosine kinase inhibitor and is now standard of care in CML patients. As used herein the term “imatinib” is used to refer to imatinib mesylate or STI-571. Although imatinib is a potent inhibitor of the kinase activity of wild type BCR-ABL, many mutant BCR-ABL isoforms are resistant to clinically achievable doses of imatinib. Clinical resistance is primarily mediated by mutations within the kinase domain of BCR-ABL and, to a lesser extent, by amplification of the BCR-ABL genomic locus (M. E. Gorre et al., Science 193, 876 (2001)). Imatinib can bind to the adenosine triphosphate (ATP)—binding site of ABL only when its activation loop is “closed” and thus the protein is in inactive conformation. This conformation-specific requirement contributes to imatinib's selectivity and the resistance shown in CML patients (N. P. Shah et al., Cancer Cell 2, 117 (2002); which is hereby incorporated herein by reference in its entirety and for all purposes). The structure and use of imatinib as an anticancer agent is described in B. J. Druker et al., N. Engl. J. Med. 344, 1031 (2001) and S. G. O'Brien et al., N. Engl. J. Med. 348, 994 (2003), both of which are incorporated herein by reference in their entirety and for all purposes.

N-(2-chloro-6-methylphenyl)-2-(6-(4-(2-hydroxyethyl)piperazin-1-yl)-2-methylpyrimidin-4-ylamino)thiazole-5-carboxamide is a synthetic small-molecule inhibitor of several SRC-family kinases, including BCR-ABL. Structural studies indicate that protein tyrosine kinase inhibitors, including N-(2-chloro-6-methylphenyl)-2-[[6-[4-(2-hydroxyethyl)-1-piperazinyl]-2-methyl-4-pyrimidinyl]amino]-5-thiazolecarboxamide, bind to the ATP-binding site in ABL, but without regard for the position of the active loop, which can be in the active or inactive conformation (B. Nagar et al., Cancer Res. 62, 4236 (2002)). The less stringent conformation requirement for binding to the ABL kinase domain is one reason N-(2-chloro-6-methylphenyl)-2-[[6-[4-(2-hydroxyethyl)-1-piperazinyl]-2-methyl-4-pyrimidinyl]amino]-5-thiazolecarboxamide retains activity against many imatinib-resistant mutants. N-(2-chloro-6-methylphenyl)-2-[[6-[4-(2-hydroxyethyl)-1-piperazinyl]-2-methyl-4-pyrimidinyl]amino]-5-different clinically relevant, imatinib-resistant BCR-ABL isoforms was successfully inhibited in the low nanomolar range (N. P. Shah et al., Science 305, 399 (2003)).

The structure and use of N-(2-chloro-6-methylphenyl)-2-[[6-[4-(2-hydroxyethyl)-1-piperazinyl]-2-methyl-4-pyrimidinyl]amino]-5-thiazolecarboxamide as an anticancer agent is described in Lombardo, L. J., et al., J. Med. Chem., 47:6658-6661 (2004) and is described in the following US patents and pending applications: U.S. Pat. No. 6,596,746, granted Jul. 22, 2003; U.S. Pat. No. 7,125,875, granted Oct. 24, 2006, all of which are incorporated by reference herein in their entirety.

In view of the resistance of certain BCR-ABL mutants to drug therapy with imatinib, there is a need for a further understanding and identification of BCR-ABL mutants that may be resistant to other kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide. The invention provided herein satisfies this and other needs.

### SUMMARY

The present inventors have discovered that mutations to the BCR-ABL polypeptide appear in certain individuals treated with N-(2-chloro-6-methylphenyl)-2-[[6-[4-(2-hydroxyethyl)-1-piperazinyl]-2-methyl-4-pyrimidinyl]amino]-5-thiazolecarboxamide and that these mutations can render the polypeptide at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide.

The present invention provides methods of identifying subjects that have mutant BCR-ABL polypeptides, and in particular, BCR-ABL polypeptides having a mutation at position 315 and/or 317. In particular, the present invention provides methods of identifying subjects that have a T315I or T315A mutation and/or a F317I, F317V, F317L, or F317S mutation. In certain aspects, the present invention provides methods of identifying subjects that have a F317I and/or T315A mutation. The invention further provides methods of identifying subject that have, not only the F3171 and/or T315A mutation, but any number of additional mutations that are associated with at least partial resistant to drug therapy, including-therapy with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof.

The present invention also provides methods of treating such subjects by tailoring their treatment regimen depending on whether or not they harbor mutant BCR-ABL

The present invention also provides methods of treating such subjects by tailoring their treatment regimen depending on whether or not they harbor mutant BCR-ABL polypeptides having a T315I or T315A mutation and/or a F3171, F317L, F317V, or F317S mutation.

The present invention also provides mutant BCR-ABL polypeptides having at least a F3171 and/or T315A mutation and polynucleotides encoding such polypeptides. The present invention further provides mutant BCR-ABL polypeptides having not only the F3171 and/or T315A mutations but any number of additional mutations that are associated with at least partial resistance to drug therapy, including therapy with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof.

Methods of identifying compounds that can be used to treat BCR-ABL related disorders are also provided herein.

Methods for determining the responsiveness of an individual to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof are provided herein. These methods can comprise the step of screening a biological sample from the individual for the presence of at least one mutation in a BCR-ABL kinase sequence wherein the presence of the mutation is indicative of the individual being at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof. In certain preferred embodiments of the present invention, the mutation is a F3171 mutation and/or a T315A mutation. In certain preferred embodiments, the mutation is a T315I or T315A mutation and/or a F317I, F317L, F317V, or F317S mutation.

Methods for treating an individual suffering from a BCR-ABL-associated disorder can comprise the steps of determining whether a biological sample obtained from the individual comprises a BCR-ABL kinase having at least one mutation, wherein the presence of the mutation is indicative of the individual being at least partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, and administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a mutation and whether or not the therapy with N-(2-chloro-6-methylphenyl)-2-[[6-[4-(2-hydroxyethyl)-1-piperazinyl]-2-methyl-4-pyrimidinyl]amino]-5-thiazolecarboxamide will be combined with a second therapy. Currently, the recommended dosage for N-(2-chloro-6-methylphenyl)-2-[[6-[4-(2-hydroxyethyl)-1-piperazinyl]-2-methyl-4-pyrimidinyl]amino]-5-thiazolecarboxamide is twice daily as a 70 mg tablet referred to as SPRYCEL™. In certain embodiments, if an individual is determined to have a BCR-ABL mutant that renders cells partially resistant to therapy with N-(2-chloro-6-methylphenyl)-2-[[6-[4-(2-hydroxyethyl)-1-piperazinyl]-2-methyl-4-pyrimidinyl]amino]-5-thiazolecarboxamide treatment, the dosage of the drug can be increased. Alternatively, the drug can be administered in combination with a second therapy for treating the BCR-ABL associated disorder. The second therapy can be any therapy effective in treating the disorder, including, for example, therapy with another protein kinase inhibitor such as imatinib, AMN107, PD180970, GGP76030, AP23464, SKI 606, NS-187, and/or AZD0530; therapy with a tubulin stabilizing agent for example, pacitaxol, epothilone, taxane, and the like; therapy with an ATP non-competitive inhibitor such as ONO12380; therapy with an Aurora kinase inhibitor such as VX-680; therapy with a p38 MAP kinase inhibitor such as BIRB-796; or therapy with a famysyl transferase inhibitor. The dosage of N-(2-chloro-6-methylphenyl)-2-[[6-[4-(2-hydroxyethyl)-1-piperazinyl]-2-methyl-4-pyrimidinyl]amino]-5-thiazolecarboxamide treatment or a pharmaceutically acceptable salt, hydrate, or solvate thereof can remain the same, be reduced, or be increased when combined with a second therapy.

The present invention provides methods for screening a biological sample, for example, a biological sample comprising cells that do not respond, or that have stopped responding, or that have a diminished response, to protein tyrosine kinase inhibitors. For example, the present invention provides a method of screening cells from an individual suffering from cancer who is being treated with imatinib and/or N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, and whose cells do not respond or have stopped responding or have a diminished response to either of the drugs, for the presence of BCR-ABL mutations described herein. The present invention provides certain BCR-ABL mutations that, if present, provide the basis upon which to alter treatment of such an individual.

An individual that is partially resistant to a protein tyrosine kinase inhibitor is an individual who has cells that have a diminished response to the protein tyrosine kinase inhibitor.

The individual to be screened or treated by the methods herein can be one that has received administration of a first kinase inhibitor to which the cancer cells in said individual have become resistant or at least partially resistant. The kinase inhibitor can be imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, another kinase inhibitor, or any combination thereof. Alternatively, the individual will have not yet had treatment with a protein kinase inhibitor. Combinations treatments comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are described in U.S. Ser. No. 10/886,955, filed Jul. 8, 2004, U.S. Ser. No. 11/265,843, filed Nov. 3, 2005, and U.S. Ser. No. 11/418,338, filed May 4, 2006, each of which are incorporated herein by reference in their entirety and for all purposes.

The invention comprises methods of establishing a treatment regimen for an individual having a BCR-ABL related disorder. The treatment regimen can comprise the administration of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof, at a higher dose or dosing frequency than recommended for an individual having non-mutated BCR-ABL or a BCR-ABL polypeptide lacking the T315A or F317I mutation. Alternatively, the treatment regiment can comprise combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide and any other agent that works to inhibit proliferation of cancerous cells or induce apoptosis of cancerous cells, including, for example, a tubulin stabilizing agent, a farnysyl transferase inhibitor, a BCR-ABL T315I inhibitor and/or another protein tyrosine kinase inhibitor. Preferred other agents include imatinib, AMN107, PD180970, CGP76030, AP23464, SKI 606, NS-187, or AZD0530. Also included are ATP non-competitive inhibitors, including, for example, ON012380, Aurora kinase inhibitors, including, fore example, VX-680, and p38 MAP kinase inhibitors, including, for example, BIRB-796. The treatment regimen can include administration of a higher dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, a reduced dose of N-(2-chloro-6-methylphenyl)-2-[[6-[4-(2-hydroxyethyl)-1-piperazinyl]-2-methyl-4-pyrimidinyl]amino]-5-thiazolecarboxamide with a second therapeutic agent, or an unchanged dose of N-(2-chloro-6-methylphenyl)-2-[[6-[4-(2

The present invention provides a kit for use in determining treatment strategy for an individual with a protein tyrosine kinase-associated disorder, comprising a means for detecting a mutant BCR-ABL kinase in a biological sample from said patient; and optionally instructions for use and interpretation of the kit results. The kit can also comprise, for example, a means for obtaining a biological sample from an individual. The treatment strategy can comprise, for example, the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. In certain embodiments, the mutant kinase will comprise a mutation at positions 315 and/or 317. In certain embodiments, the mutation at position 315 will be a T315I or T315A mutation and the mutation at position 317 will be a F317I, F317V, F317L, or F317S mutation.

The present invention also provides methods of identifying amino acid positions within the BCR-ABL polypeptide that may confer at least partial resistance to a tyrosine kinase inhibitor. The methods can comprise the steps of creating a co-crystal of the polypeptide with the BCR-ABL inhibitor, and identifying the amino acid positions of the polypeptide that either contact, bond, interface, or interact with the BCR-ABL inhibitor. In certain embodiments, the contact or interface amino acids will be at positions 248, 299, 315, and 317. In certain embodiments, the contacting, bonding, interfacing, or interacting amino acids, or amino acids that stabilize the contacting, bonding, interfacing, or interacting amino acids will be at positions 244, 248, 255, 290, 299, 313, 315, 316, 317, 318, 320, 321 and 380.

The F317I mutation and/or the T315A mutation of a BCR-ABL protein can be indicative of a greater likelihood of having partial resistance to therapy with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof. Additional mutations may be present as well, including for example any combination of the mutations described herein, i.e., E279K, F359C, F3591, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, D381G, F382L, L387M, M388L, T389S, T392A, T394A, A395G, H396K, H396R, A399G, P402T, T406A, S417Y, and F486S, including for example, M244V, G250E, Q252H, Q252R, Y253F, Y253H, E255K, E255V, T315I, F317L, M351T, E355G, F359V, H396R, F486S; M244V, E279K, F359C, F359I, L364I, L387M, F486S and any combination thereof; and L248R, Q252H, E255K, V299L, T315I, F317V, F317L, F317S and any combination thereof.

The present invention not only provides screening and diagnostic methods but also polynucleotides encoding a BCR-ABL mutant polypeptide having substantial identity or exact identity to wild type BCR-ABL except for the presence of at least one of the F317I mutation or the T315A mutation, and fragments thereof. The polynucleotide can encode a BCR-ABL mutant polypeptide having the F3171 mutation and/or the T315A mutation and any combination of the additional mutations described herein. Also provided are BCR-ABL mutant polypeptides having substantial identity or exact identity to SEQ ID NO:2 except for the presence of at least one of the F3171 mutation or the T315A mutation, and fragments thereof. The polypeptides of the present invention can have the 317I mutation and/or the T315A mutation and any combination of the additional mutations described herein. Antibodies directed to the mutant BCR-ABL polypeptides and methods of using the antibodies to detect the polypeptides are also included herein.

Methods of identifying compounds that bind to the mutant polypeptides described herein can comprise the steps of contacting a test compound with the mutant BCR-ABL polypeptide and determining whether the mutant polypeptide specifically binds to the test compound.

Methods of determining whether a test compound modulates, i.e., inhibits, the tyrosine kinase activity of a mutant BCR-ABL polypeptide can comprise the steps of obtaining mammalian cells transfected with a construct encoding the mutant BCR-ABL polypeptide, contacting the cells with the test compound, and monitoring the cells for tyrosine kinase activity of the mutant BCR-ABL polypeptide wherein a modulation, i.e., inhibition, in tyrosine kinase activity in the presence of the test compound identifies the test compound as a modulator, i.e., inhibitor, of the mutant BCR-ABL polypeptides.

### BRIEF DESCRIPTION OF THE DRAWINGS

FIG. 1: This FIGURE shows BCR-ABL mutations present before and after treatment with N-(2-chloro-6-methylphenyl)-2-[[6-[4-(2-hydroxyethyl)-1-piperazinyl]-2-methyl-4-pyrimidinyl]amino]-5-thiazolecarboxamide. Two mutations, in particular, became visible after

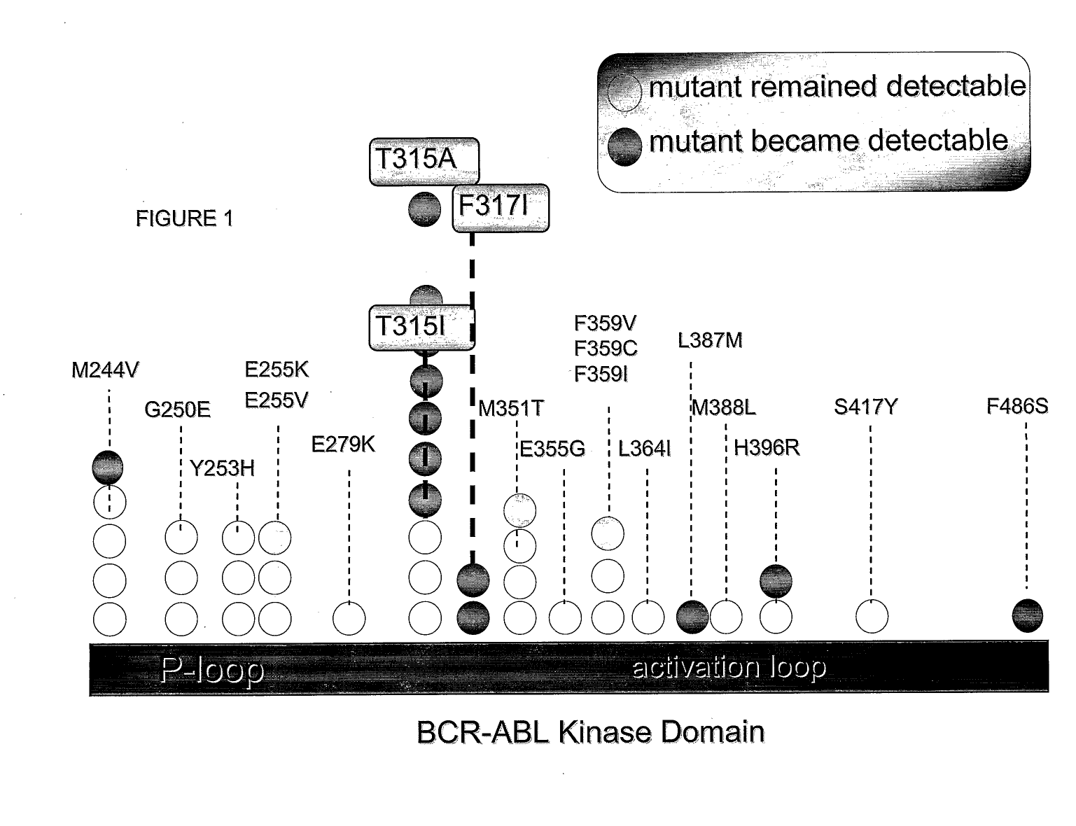

### DETAILED DESCRIPTION OF ILLUSTRATIVE EMBODIMENTS

### I. Introduction

The present invention is based, in part, on the discovery that certain individuals treated with N-(2-chloro-6-methylphenyl)-2-(6-(4-(2-hydroxyethyl)piperazin-1-yl)-2-methylpyrimidin-4-ylamino)thiazole-5-carboxamide develop mutations at select amino acid positions within the BCR-ABL kinase domain and that these mutations are associated with at least partial resistance to therapy with N-(2-chloro-6-methylphenyl)-2-(6-(4-(2-hydroxyethyl)piperazin-1-yl)-2-methylpyrimidin-4-ylamino)thiazole-5-carboxamide.

Recognition that these mutations exist in an individual having a BCR-ABL-associated disorder can, among other things, help in determining the responsiveness of individuals to treatment with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, and it can help tailor treatment regimens appropriately.

As is known in the art, N-(2-chloro-6-methylphenyl)-2-[[6-[4-(2-hydroxyethyl)-1-piperazinyl]-2-methyl-4-pyrimidinyl]amino]-5-thiazolecarboxamide refers to a compound having the following structure (I):

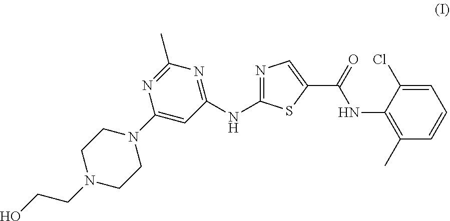

Compound (I) can also be referred to as N-(2-chloro-6-methylphenyl)-2-((6-(4-(2-hydroxyethyl)-1-piperazinyl)-2-methyl-4-pyrimidinyl)amino)-1,3-thiazole-5-carboxamide in accordance with IUPAC nomenclature. Use of the term “N-(2-chloro-6-methylphenyl)-2-[[6-[4-(2-hydroxyethyl)-1-piperazinyl]-2-methyl-4-pyrimidinyl]amino]-5-thiazolecarboxamide” encompasses (unless otherwise indicated) solvates (including hydrates) and polymorphic forms of the compound (I) or its salts (such as the monohydrate form of (I) described in U.S. Ser. No. 11/051,208, filed Feb. 4, 2005, incorporated herein by reference in its entirety and for all purposes). Pharmaceutical compositions of N-(2-chloro-6-methylphenyl)-2-[[6-[4-(2-hydroxyethyl)-1-piperazinyl]-2-methyl-4-pyrimidinyl]amino]-5-thiazolecarboxamide and one or more diluents, vehicles and/or excipients, such as those compositions described in U.S. Ser. No. 11/402,502, filed Apr. 12, 2006, incorporated herein by reference in its entirety and for all purposes. One example of a pharmaceutical composition comprising N-(2-chloro-6-methylphenyl)-2-[[6-[4-(2-hydroxyethyl)-1-piperazinyl]-2-methyl-4-pyrimidinyl]amino]-5-thiazolecarboxamide is SPRYCEL™ (Bristol-Myers Squibb Company). SPRYCEL™ comprises N-(2-chloro-6-methylphenyl)-2-[[6-[4-(2-hydroxyethyl)-1-piperazinyl]-2-methyl-4-pyrimidinyl]amino]-5-thiazolecarboxamide as the active ingredient, also referred to as dasatinib, and as inactive ingredients or excipients, lactose monohydrate, microcrystalline cellulose, croscarmellose sodium, hydroxypropyl cellulose, and magnesium stearate in a tablet comprising hypromellose, titanium dioxide, and polyethylene glycol.

It is to be understood that this invention is not limited to particular methods, reagents, compounds, compositions, or biological systems, which can, of course, vary. It is also to be understood that the terminology used herein is for the purpose of describing particular aspects only, and is not intended to be limiting. As used in this specification and the appended claims, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

### II. Polypeptides, Polynucleotides, and Antibodies

The present invention provides isolated novel BCR-ABL nucleotides and their encoded proteins having mutations at certain amino acids that can render an individual at least acceptable salt of hydrate thereof. At least one of the mutations is a F317I or T315A mutation.

The single letter amino acid sequence of wild-type human BCR-ABL protein shown is known in the art and provided as SEQ ID NO:2. The nucleic acid sequence of BCR-ABL is also provided herein as SEQ ID NO:1. Wild-type and variant sequences can also be found in GenBank database. See for example, accession number gi|177943 (encoded by gi|177942), NP_{—}005148.1, and NP_{—}005148.2.

For purposes of shorthand designation of the mutant variants described herein, it is noted that numbers refer to the amino acid residue position along the amino acid sequence of the BCR-ABL polypeptide as provided in SEQ ID NO:2 of the sequence listing (i.e., wild-type BCR-ABL polypeptide). For example, F317 refers to the amino acid phenylalanine at position 317. Amino acid substitutions at a particular position are written as the wild type amino acid, position number, and amino acid substituted therein, in that order. For example, F317I refers to a substitution of isoleucine for phenylalanine at position 317. Similarly, T315A refers to an alanine for threonine substitution at position 315. Amino acid identification uses the single-letter alphabet of amino acids, as shown in Table 1 below.

TABLE 1

| | | | | | |
|---|---|---|---|---|---|
| Asp | D | Aspartic acid | Ile | I | Isoleucine |
| Thr | T | Threonine | Leu | L | Leucine |
| Ser | S | Serine | Tyr | Y | Tyrosine |
| Glu | E | Glutamic acid | Phe | F | Phenylalanine |
| Pro | P | Proline | His | H | Histidine |
| Gly | G | Glycine | Lys | K | Lysine |
| Ala | A | Alanine | Arg | R | Arginine |
| Cys | C | Cysteine | Trp | W | Tryptophan |
| Val | V | Valine | Gln | Q | Glutamine |
| Met | M | Methionine | Asn | N | Asparagine |

Accordingly the present invention provides isolated novel BCR-ABL polypeptides comprising the amino acid sequence set forth as SEQ ID NO:2 or having substantial identity to the amino acid sequence set forth as SEQ ID NO:2 and having at least a mutations or any combination thereof: E279K, F359C, F359I, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V3791, A380T, A380V, D381G, F382L, L387M, M388L, T389S, T392A, T394A, A395G, H396K, H396R, A399G, P402T, T406A, S417Y, or F486S, including for example, M244V, G250E, Q252H, Q252R, Y253F, Y253H, E255K, E255V, T315I, F317L, M351T, E355G, F359V, H396R, F486S; M244V, E279K, F359C, F359I, L364I, L387M, F486S and any combination thereof; and L248R, Q252H, E255K, V299L, T315I, F317V, F317L, F317S and any combination thereof.

The present invention also provides conservatively modified variants of SEQ ID NO:2 having at least a F317I and/or T315A mutation, and fragments thereof.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences. As to a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “polynucleotide” means a polymeric form of nucleotides of at least about 10 bases or base pairs in length, either ribonucleotides or deoxynucleotides or a modified form of either type of nucleotide, and is meant to include single and double stranded forms of DNA.

As used herein, a polynucleotide is said to be “isolated” when it is substantially separated from contaminant polynucleotides that correspond or are complementary to genes other than, the BcrAbl gene or mutants thereof. As used herein, a polypeptide is said to be “isolated” when it is substantially separated from contaminant polypeptide that correspond to polypeptides other than the BCRABL peptide or mutant polypeptides or fragments thereof. A skilled artisan can readily employ polynucleotide or polypeptide isolation procedures well known in the art to obtain said isolated polynucleotides and/or polypeptides.

As used herein “substantial identity” to a specified sequence refers to 80% identity or greater, i.e., 81%, 82%, 83%, 84%, 85%, 86%, 87%, 88%, 89%, 90%, 91%, 91%, 93%, 94%, 95%, 96%, 97%, 98%, o99%, 99.5% or 99.9% identity to the specified sequence.

In the context of amino acid sequence comparisons, the term “identity” is used to identify and express the percentage of amino acid residues at the same relative positions that similar, using the conserved amino acid criteria of BLAST analysis, as is generally understood in the art. For example, identity and homology values can be generated by WU-BLAST-2 (Altschul et al., Methods in Enzymology, 266: 460-480 (1996): http://blast.wustl/edu/b-last/README.html).

“Percent (%) amino acid sequence identity” with respect to the sequences identified herein is defined as the percentage of amino acid residues in a candidate sequence that are identical with the amino acid residues in the BCR-ABL sequence, after aligning the sequences and introducing gaps, if necessary, to achieve the maximum percent sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent ammo acid identity values can also be obtained using the sequence comparison computer program, ALIGN-2, the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

The polynucleotides of the invention are useful for a variety of purposes, including, for example, their use in the detection of the gene(s), mRNA(s), or fragments thereof; as reagents for the diagnosis and/or prognosis of BCR-ABL associated disorders, including cancers; as coding sequences capable of directing the expression of their encoded polypeptides; and as tools for modulating or inhibiting the function of the encoded protein.

Further specific embodiments of this aspect of the invention include primers and primer pairs, which allow the specific amplification of the polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers can be used to detect the presence of a polynucleotide of the present invention in a sample and as a means for detecting a cell expressing a protein of the present invention.

As used herein, the terms “hybridize”, “hybridizing”, “hybridizes” and the like, used in the context of polynucleotides, are meant to refer to conventional hybridization 0.1×SSC/0.1% SDS are above 55° C., and most preferably to stringent hybridization conditions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, are known to those of skill in the art and as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

“Moderately stringent conditions” can be identified as described by Sambrook et al., 1989, Molecular Cloning: A Laboratory Manual, New York: Cold Spring Harbor Press, and include the use of washing solution and hybridization conditions (e.g., temperature, ionic strength and % SDS) less stringent than those described above. A non-limiting example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will

The invention also provides recombinant DNA or RNA molecules comprising a polynucleotide of the present invention, including, for example, phages, plasmids, phagemids, cosmids, YACs, BACs, as well as various viral and non-viral vectors well known in the art, and cells transformed or transfected with such recombinant DNA or RNA molecules. As used herein, a recombinant DNA or RNA molecule is a DNA or RNA molecule that has been subjected to molecular manipulation in vitro. Methods for generating such molecules are well known (see, for example, Sambrook et al, 1989, supra).

The invention further provides a host-vector system comprising a recombinant DNA molecule containing a polynucleotide of the present invention within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 cell). Examples of suitable mammalian cells include various cancer cell lines, other transfectable or transducible cell lines, including those mammalian cells routinely used for the expression of recombinant proteins (e.g., COS, CHO, 293, 293T cells and the like). More particularly, a polynucleotide encoding a mutant BCR-ABL of the present invention can be used to generate proteins or fragments thereof using any number of host vector systems routinely used and widely known in the art. Cell lines comprising the BCR-ABL polypeptides and BCR-ABL polynucleotides of the present invention are provided herein.

Proteins encoded by the genes of the present invention, or by fragments thereof, have a variety of uses, including, for example, generating antibodies and in methods for identifying ligands and other agents (e.g. small molecules such as 2-phenylpyrimidines) and cellular constituents that bind to a gene product. Antibodies raised against a BCR-ABL mutant protein or fragment thereof are useful in diagnostic and prognostic assays, imaging methodologies (including, particularly, cancer imaging), and therapeutic methods in the management of human cancers characterized by expression of a protein of the present invention, including, for example, cancer of the lymphoid lineages. Various immunological assays useful for the detection of proteins of the present invention are contemplated, including, for example, various types of radioimmunoassays, enzyme-linked immunosorbent assays (ELISA), enzyme-linked immunofluorescent assays (ELIFA), immunocytochemical methods, and the like. Such antibodies can be labeled and used as immunological imaging reagents capable of detecting leukemia cells (e.g., in radioscintigraphic imaging methods).

Current Protocols in Molecular Biology, 1995, supra). Vectors for mammalian expression include, for example, pcDNA 3.1 myc-His-tag (Invitrogen) and the retroviral vector pSR.alpha.tkneo (Muller et al., 1991, MCB 11:1785). Using these expression vectors, the polypeptides of the present invention can be preferably expressed in cell lines, including for example CHO COS, 293, 293T, rat-1, 3T3 etc. The host vector systems of the invention are useful for the production of a mutant protein or fragment thereof. Such host-vector systems can be employed to study the functional properties of the proteins.

The present invention provides antibodies that can specifically bind with the polypeptides of the present invention. The term “antibody” is used in the broadest sense and specifically covers monoclonal antibodies, polyclonal antibodies, antibody compositions with polyepitopic specificity, bispecific antibodies, diabodies, chimeric, single-chain, and humanized antibodies, as well as antibody fragments (e.g., Fab, F(ab′)₂, and Fv), so long as they exhibit the desired biological activity. Antibodies can be labeled for use in biological assays (e.g., radioisotope labels, fluorescent labels) to aid in detection of the antibody.

Antibodies that bind to mutant polypeptides can be prepared using, for example, intact polypeptides or fragments containing small peptides of interest, which can be prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, for example, bovine serum albumin (BSA), keyhole limpet hemocyanin (KLH), and thyroglobulin. The coupled peptide is then used to immunize the animal (e.g, a mouse, a rat, or a rabbit).

The term “antigenic determinant” refers to that portion of a molecule that makes contact with a particular antibody (i.e., an epitope). When a protein or fragment of a protein is used to immunize a host animal, numerous regions of the protein can induce the production of antibodies which bind specifically to a given region or three-dimensional structure on the protein; each of these regions or structures is referred to as an antigenic determinant. An antigenic determinant can compete with the intact antigen (i.e., the immunogen used to elicit the immune response) for binding to an antibody.

The phrase “specifically binds to” refers to a binding reaction which is determinative of the presence of a target in the presence of a heterogeneous population of other biologics. Thus, under designated assay conditions, the specified binding region bind moiety that is selected for its specificity for a particular target. A variety of assay formats can be used to select binding regions that are specifically reactive with a particular analyte. Typically a specific or selective reaction will be at least twice background signal or noise and more typically more than 10 times background. For purposes of the present invention, compounds, for example small molecules, can be considered for their ability to specifically bind to mutants described herein.

### III. Exemplary Indications, Conditions, Diseases, and Disorders

The present invention provides methods of determining responsiveness of an individual having a BCR-ABL associated disorder to a certain treatment regimen and methods of treating an individual having a BCR-ABL associated disorder.

The term “BCR-ABL” as used herein is inclusive of both wild-type and mutant BCR-ABL.

“BCR-ABL associated disorders” are those disorders which result from BCR-ABL activity, including mutant BCR-ABL activity, and/or which are alleviated by the inhibition of BCR-ABL, including mutant BCR-ABL, expression and/or activity. A reciprocal translocation between chromosomes 9 and 22 produces the oncogenic BCR-ABL fusion protein. The phrase “BCR-ABL associated disorders” is inclusive of “mutant BCR-ABL associated disorders”.

Disorders included in the scope of the present invention include, for example, leukemias, including, for example, chronic myeloid leukemia, acute lymphoblastic leukemia, and Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia, chronic lymphocytic leukemia, mastocytosis and any symptom associated with mastocytosis. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a included within the scope of protein tyrosine kinase-associated disorders including, for example, the following: carcinoma, including that of the bladder, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma. In certain preferred embodiments, the disorder is leukemia, breast cancer, prostate cancer, lung cancer, colon cancer, melanoma, or solid tumors. In certain preferred embodiments, the leukemia is chronic myeloid leukemia (CML), Ph+ ALL, AML, imatinib-resistant CML, imatinib-intolerant CML, accelerated CML, lymphoid blast phase CML,

A “solid tumor” includes, for example, sarcoma, melanoma, carcinoma, or other solid tumor cancer.

The terms “cancer”, “cancerous”, or “malignant” refer to or describe the physiological condition in mammals that is typically characterized by unregulated cell growth. Examples of cancer include, for example, leukemia, lymphoma, blastoma, carcinoma and sarcoma. More particular examples of such cancers include chronic myeloid leukemia, acute lymphoblastic leukemia, Philadelphia chromosome positive acute lymphoblastic leukemia (Ph+ ALL), squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, pediatric sarcoma, sinonasal natural killer, multiple myeloma, acute myelogenous leukemia (AML), and chronic lymphocytic leukemia (CML).

and their precursors in the blood and bone marrow. Leukemia is generally clinically classified on the basis of (1) the duration and character of the disease—acute or chronic; (2) the type of cell involved; myeloid (myelogenous), lymphoid (lymphogenous), or monocytic; and (3) the increase or non-increase in the number of abnormal cells in the blood—leukemic or aleukemic (subleukemic). Leukemia includes, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia. In certain aspects, the present invention provides treatment for chronic myeloid leukemia, acute lymphoblastic leukemia, and/or Philadelphia chromosome positive acute lymphoblastic leukemia (Ph+ ALL).

A “mutant BCR-ABL” encompasses a BCR-ABL tyrosine kinase with an amino acid sequence that differs from wild type BCR-ABL tyrosine kinase by one or more amino acid substitutions, additions or deletions. For example a substitution of the amino acid at position 317 with another amino acid would result in a mutant BCR-ABL tyrosine kinase.

“Mutant BCR-ABL associated disorder” is used to describe a BCR-ABL associated disorder in which the cells involved in said disorder are or become resistant to treatment with a kinase inhibitor used to treat said disorder as a result of a mutation in BCR-ABL. For example, a kinase inhibitor compound can be used to treat a cancerous condition, which compound inhibits the activity of wild type BCR-ABL which will inhibit proliferation and/or induce apoptosis of cancerous cells. Over time, a mutation can be introduced into the gene encoding BCR-ABL kinase, which can alter the amino acid sequence of the BCR-ABL kinase and cause the cancer cells to become resistant, or at least partially resistant, to treatment with the compound. Alternatively, a mutation can already be present within the gene encoding propensity to differentiate into a cancerous or proliferative state, and also result in these cells being less sensitive to treatment with a protein tyrosine kinase inhibitor. Such situations are expected to result, either directly or indirectly, in a “mutant BCR-ABL kinase associated disorder” and treatment of such condition will require a compound that is at least partially effective against the mutant BCR-ABL, preferably against both wild type BCR-ABL and the mutant BCR-ABL. In the instance where an individual develops at least partial resistance to the kinase inhibitor imatinib, the mutant BCR-ABL associated disorder is one that results from an imatinib-resistant BCR-ABL mutation, or a protein tyrosine kinase inhibitor resistant BCR-ABL mutation. Similarly, in the instance where an individual develops at least partial resistance to the kinase inhibitor N-(2-chloro-6-methylphenyl)-2-[[6-[4-(2-hydroxyethyl)-1-piperazinyl]-2-methyl-4-pyrimidinyl]amino]-5-thiazolecarboxamide, the mutant BCR-ABL associated disorder is one that results from an N-(2-chloro-6-methylphenyl)-2-[[6-[4-(2-hydroxyethyl)-1-piperazinyl]-2-methyl-4-pyrimidinyl]amino]-5-thiazolecarboxamide resistant BCR-ABL mutation, or a protein tyrosine kinase inhibitor resistant BCR-ABL mutation. The present inventors discovered that after treatment with N-(2-chloro-6-methylphenyl)-2-[[6-[4-(2-hydroxyethyl)-1-piperazinyl]-2-methyl-4-pyrimidinyl]amino]-5-thiazolecarboxamide, certain individuals developed F3171 and/or T315A mutations. The present invention provides, among other things, methods of treating mutant BCR-ABL associated disorders and methods of identifying if an individual has a mutant BCR-ABL associated disorder.

“Imatinib-resistant BCR-ABL mutation” refers to a specific mutation in the amino acid sequence of BCR-ABL that confers upon cells that express said mutation resistance to treatment with imatinib. As discussed herein such mutations can include mutations at the T315I position of BCR-ABL. Additional mutations that may render a BCR-ABL protein at least partially imatinib resistant can include, for example, E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L3641, E373K, N374D, K378R, Number 20030158105, incorporated herein by reference in its entirety and for all purposes).

“N-(2-chloro-6-methylphenyl)-2-[[6-[4-(2-hydroxyethyl)-1-piperazinyl]-2-methyl-4-pyrimidinyl]amino]-5-thiazolecarboxamide-resistant BCR-ABL mutation” refers to a specific mutation in the amino acid sequence of BCR-ABL that confers upon cells that express said mutation resistance to treatment with N-(2-chloro-6-methylphenyl)-2-[[6-[4-(2-hydroxyethyl)-1-piperazinyl]-2-methyl-4-pyrimidinyl]amino]-5-thiazolecarboxamide. As discussed herein such mutations can include the F3171 and T315A mutations. Additional mutations that render a BCR-ABL protein at least partially N-(2-chloro-6-methylphenyl)-2-[[6-[4-(2-hydroxyethyl)-1-piperazinyl]-2-methyl-4-pyrimidinyl]amino]-5-thiazolecarboxamide resistant include, for example, T315I.

“Imatinib-resistant CML” refers to a CML in which the cells involved in CML are resistant to treatment with imatinib. Generally it is a result of a mutation in BCR-ABL.

“Imatinib-intolerant CML” refers to a CML in which the individual having the CML is intolerant to treatment with imatinib, i.e., the toxic and/or detrimental side effects of imatinib outweigh any therapeutically beneficial effects.

### IV. Detection Methods

The invention provides methods of screening a biological sample from an individual for the presence of at least one mutation in the BCR-ABL kinase sequence, as well as methods for identifying a cell that expresses mutant BCR-ABL kinase.

Methods of identifying the amino acid and nucleic acid sequence of a wild-type or mutant BCR-ABL polynucleotide or BCR-ABL polypeptide are known in the art. Standard molecular biology techniques are contemplated for precisely determining a BCR-ABL mutation in the cells of a given individual.

Antibodies that immunospecifically bind to a mutant BCR-ABL kinase can be used in identifying one or more of the BCR-ABL mutants described herein. Contemplated herein are antibodies that specifically bind to a mutant BCR-ABL kinase of the present invention and that do not bind (or bind weakly) to wild type BCR-ABL protein or polypeptides. Anti-mutant BCR-ABL kinase antibodies include, for example, monoclonal and polyclonal antibodies as well as fragments containing the antigen binding domain and/or one or more complementarity determining regions of these antibodies.

particular structural domain. For example, antibodies useful for diagnostic purposes can be those which react with an epitope in a mutated region of the BCR-ABL protein as expressed in cancer cells. For example, antibodies that bind specifically to a F317I and/or T315A mutant BCR-ABL kinase. Such antibodies can be generated by using the mutant BCR-ABL kinase protein described herein, or using peptides derived from various domains thereof, as an immunogen.

Mutant BCR-ABL kinase antibodies of the invention can be particularly useful in cancer (e.g., chronic myeloid leukemia, acute lymphoblastic leukemia, Philadelphia chromosome positive acute lymphoblastic leukemia (Ph+ ALL, GIST)) therapeutic strategies, diagnostic and prognostic assays, and imaging methodologies. Similarly, such antibodies can be useful in the diagnosis, and/or prognosis of other cancers, to the extent such mutant BCR-ABL kinase is also expressed or overexpressed in other types of cancer. The invention provides various immunological assays useful for the detection and quantification of mutant BCR-ABL kinase proteins and polypeptides. Such assays generally comprise one or more mutant BCR-ABL kinase antibodies capable of recognizing and binding a mutant BCR-ABL kinase protein, as appropriate, and can be performed within various immunological assay formats well known in the art, including, for example, various types of radioimmunoassays, enzyme-linked immunosorbent assays (ELISA), enzyme-linked immunofluorescent assays (ELIFA), and the like. In addition, immunological imaging methods capable of detecting cancer cells are also provided by the invention including, for example, imaging methods using labeled mutant BCR-ABL kinase antibodies. Such assays can be used clinically in the detection, monitoring, and prognosis of cancers.

Accordingly, the present invention provides methods of assaying for the presence of a mutant BCR-ABL polypeptide of the present invention. By way of example only, in certain embodiments, an antibody raised against the fragment, or other binding moiety capable of specifically binding to the target analyte, is immobilised onto a solid substrate to form a first complex and a biological test sample from a patient is brought into contact with the bound molecule. After a suitable period of incubation, for a period of time sufficient to allow formation of an antibody-secondary complex, a second antibody labelled with a reporter molecule capable of producing a detectable signal is then added and incubated, allowing sufficient time for the formation of a tertiary complex. Any unreacted material is washed away, and the presence of the tertiary complex is determined by observation of a signal produced by the reporter molecule. The results may either be qualitative, by simple observation of the visible signal or may be antibody are added simultaneously to the bound antibody, or a reverse assay in which the labelled antibody and sample to be tested are first combined, incubated and then added simultaneously to the bound antibody. These techniques are well known to those skilled in the art, and the possibility of variations will be readily apparent.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecule in this type of assay are either enzymes, fluorophores or radionuclide containing molecules (i.e. radioisotopes).

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of cross-linking covalently binding or physically adsorbing the molecule to the insoluble carrier.

The expression profiles of mutant BCR-ABL kinases can be used as diagnostic markers for disease states. The status of mutant BCR-ABL kinase gene products in patient samples can be analyzed by a variety protocols that are well known in the art including the following non-limiting types of assays: PCR-free genotyping methods, Single-step homogeneous methods, Homogeneous detection with fluorescence polarization, Pyrosequencing, “Tag” based DNA chip system, Bead-based methods, fluorescent dye chemistry, Mass spectrometry based genotyping assays, TaqMan genotype assays, Invader genotype assays, microfluidic genotype assays, immunohistochemical analysis, the variety of Northern blotting techniques including in situ hybridization, RT-PCR analysis (for example on laser capture micro-dissected samples), western blot analysis, tissue array analysis, and any other methods known in the art or described elsewhere herein.

Specifically encompassed by the present invention are the following, non-limiting genotyping methods: Landegren, U., Nilsson, M. & Kwok, P. Genome Res 8, 769-776 (1998); Kwok, P., Pharmacogenomics 1, 95-100 (2000); Gut, I., Hum Mutat 17, 475-492 (2001); Whitcombe, D., Newton, C. & Little, S., Curr Opin Biotechnol 9, 602-608 (1998); Tillib, S. & Mirzabekov, A., Curr Opin Biotechnol 12, 53-58 (2001); Winzeler, E. et al., Science 281, 1194-1197 (1998); Lyamichev, V. et al., Nat Biotechnol 17, 292-296 (1999); Hall, J. et al., Proc Natl (1994); Baner, J., Nilsson, M., Mendel-Hartvig, M. & Landegren, U., Nucleic Acids Res 26, 5073-5078 (1998); Baner, J. et al., Curr Opin Biotechnol 12, 11-15 (2001); Hatch, A., Sano, T., Misasi, J. & Smith, C., Genet Anal 15, 35-40 (1999); Lizardi, P. et al., Nat Genet. 19, 225-232 (1998); Zhong, X., Lizardi, P., Huang, X., Bray-Ward, P. & Ward, D., Proc Natl Acad Sci U S A 98, 3940-3945 (2001); Faruqi, F. et al. BMC Genomics 2, 4 (2001); Livak, K., Gnet Anal 14, 143-149 (1999); Marras, S., Kramer, F. & Tyagi, S., Genet Anal 14, 151-156 (1999); Ranade, K. et al., Genome Res 11, 1262-1268 (2001); Myakishev, M., Khlipin, Y., Hu, S. & Hamer, D., Genome Re 11, 163-169 (2001); Beaudet, L., Bedard, J., Breton, B., Mercuri, R. & Budarf, M., Genome Res 11, 600-608 (2001); Chen, X., Levine, L. & PY, K., Genome Res 9, 492-498 (1999); Gibson, N. et al., Clin Chem 43, 1336-1341 (1997); Latif, S., Bauer-Sardina, I., Ranade, K., Livak, K. & PY, K., Genome Res 11, 436-440 (2001); Hsu, T., Law, S., Duan, S., Neri, B. & Kwok, P., Clin Chem 47, 1373-1377 (2001); Alderborn, A., Kristofferson, A. & Hammerling, U., Genome Res 10, 1249-1258 (2000); Ronaghi, M., Uhlen, M. & Nyren, P., Science 281, 363, 365 (1998); Ronaghi, M., Genome Res 11, 3-11 (2001); Pease, A. et al., Proc Natl Acad Sci U S A 91, 5022-5026 (1994); Southern, E., Maskos, U. & Elder, J., Genomics 13, 1008-1017 (1993); Wang, D. et al., Science 280, 1077-1082 (1998); Brown, P. & Botstein, D., Nat Genet. 21, 33-37 (1999); Cargill, M. et al. Nat Genet. 22, 231-238 (1999); Dong, S. et al., Genome Res 11, 1418-1424 (2001); Halushka, M. et al., Nat Genet. 22, 239-247 (1999); Hacia, J., Nat Genet. 21, 42-47 (1999); Lipshutz, R., Fodor, S., Gingeras, T. & Lockhart, D., Nat Genet. 21, 20-24 (1999); Sapolsky, R. et al., Genet Anal 14, 187-192 (1999); Tsuchihashi, Z. & Brown, P., J Virol 68, 5863 (1994); Herschlag, D., J Biol Chem 270, 20871-20874 (1995); Head, S. et al., Nucleic Acids Res 25, 5065-5071 (1997); Nikiforov, T. et al., Nucleic Acids Res 22, 4167-4175 (1994); Syvanen, A. et al., Genomics 12, 590-595 (1992); Shumaker, J., Metspalu, A. & Caskey, C., Hum Mutat 7, 346-354 (1996); Lindroos, K., Liljedahl, U., Raitio, M. & Syvanen, A., Nucleic Acids Res 29, E69-9 (2001); Lindblad-Toh, K. et al., Nat Genet. 24, 381-386 (2000); Pastinen, T. et al., Genome Res 10, 1031-1042 (2000); Fan, J. et al., Genome Res 10, 853-860 (2000); Hirschhorn, J. et al., Proc Natl Acad Sci USA 97, 12164-12169 (2000); Bouchie, A., Nat Biotechnol 19, 704 (2001); Hensel, M. et al., Science 269, 400-403 (1995); Shoemaker, D., Lashkari, D., Morris, D., Mittmann, M. & Davis, R. Nat Genet. 14, 450-456 (1996); Gerry, N. et al., J Mol Biol 292, 251-262 (1999); Ladner, D. et al., Lab Invest 81, 1079-1086 (2001); lannone, M. et al., Cytometry 39, 131-140 (2000); Fulton, R., McDade, R., Smith, P., Kienker, L. & Kettman, J. J., Clin Chem 43, 1749-1756 (1997); Armstrong, B., Stewart, M. & K., Taylor, L., Schultz, S. & Walt, D., Anal Chem 70, 1242-1248 (1998); Steemers, F., Ferguson, J. & Walt, D., Nat Biotechnol 18, 91-94 (2000); Chan, W. & Nie, S., Science 281, 2016-2018 (1998); Han, M., Gao, X., Su, J. & Nie, S., Nat Biotechnol 19, 631-635 (2001); Griffin, T. & Smith, L., Trends Biotechnol 18, 77-84 (2000); Jackson, P., Scholl, P. & Groopman, J., Mol Med Today 6, 271-276 (2000); Haff, L. & Smimov, I., Genome Res 7, 378-388 (1997); Ross, P., Hall, L., Smimov, I. & Haff, L., Nat Biotechnol 16, 1347-1351 (1998); Bray, M., Boerwinkle, E. & Doris, P. Hum Mutat 17, 296-304 (2001); Sauer, S. et al., Nucleic Acids Res 28, E13 (2000); Sauer, S. et al., Nucleic Acids Res 28, E100 (2000); Sun, X., Ding, H., Hung, K. & Guo, B., Nucleic Acids Res 28, E68 (2000); Tang, K. et al., Proc Natl Acad Sci USA 91, 10016-10020 (1999); Li, J. et al., Electrophoresis 20, 1258-1265 (1999); Little, D., Braun, A., O'Donnell, M. & Koster, H., Nat Med 3, 1413-1416 (1997); Little, D. et al. Anal Chem 69, 4540-4546 (1997); Griffin, T., Tang, W. & Smith, L., Nat Biotechnol 15, 1368-1372 (1997); Ross, P., Lee, K. & Belgrader, P., Anal Chem 69, 4197-4202 (1997); Jiang-Baucom, P., Girard, J., Butler, J. & Belgrader, P., Anal Chem 69, 4894-4898 (1997); Griffin, T., Hall, J., Prudent, J. & Smith, L., Proc Natl Acad Sci USA 96, 6301-6306 (1999); Kokoris, M. et al., Mol Diagn 5, 329-340 (2000); Jurinke, C., van den Boom, D., Cantor, C. & Koster, H. (2001); and/or Taranenko, N. et al., Genet Anal 13, 87-94 (1996), all of which are incorporated herein by reference in their entirety.

The following additional genotyping methods are also encompassed by the present invention: the methods described and/or claimed in U.S. Pat. No. 6,458,540, incorporated herein by reference in its entirety; and the methods described and/or claimed in U.S. Pat. No. 6,440,707, incorporated herein by reference in its entirety.

Probes and primers can be designed so as to be specific to such mutation analysis and can be derived from the wild type BCR-ABL sequence, segments and complementary sequences thereof.

Additionally, the invention provides assays for the detection of mutant BCR-ABL kinase polynucleotides in a biological sample, such as cell preparations, and the like. A number of methods for amplifying and/or detecting the presence of mutant BCR-ABL kinase polynucleotides are well known in the art and can be employed in the practice of this aspect of the invention.

In certain embodiments, a method for detecting a mutant BCR-ABL kinase mRNA in a biological sample comprises producing cDNA from the sample by reverse cDNAs therein; and detecting the presence of the amplified mutant BCR-ABL kinase cDNA. Any number of appropriate sense and antisense probe combinations can be designed from the nucleotide sequences provided for a mutant BCR-ABL kinase and used for this purpose.

The invention also provides assays for detecting the presence of a mutant BCR-ABL kinase protein in a biological sample. Methods for detecting a mutant BCR-ABL kinase protein are also well known and include, for example, immunoprecipitation, immunohistochemical analysis, Western Blot analysis, molecular binding assays, ELISA, ELIFA and the like. For example, in one embodiment, a method of detecting the presence of a mutant BCR-ABL kinase protein in a biological sample comprises first contacting the sample with a BCR-ABL antibody, a mutant BCR-ABL kinase-reactive fragment thereof, or a recombinant protein containing an antigen binding region of a mutant BCR-ABL kinase antibody; and then detecting the binding of mutant BCR-ABL kinase protein in the sample thereto.

Methods for identifying a cell that expresses mutant BCR-ABL kinase are also provided. In one embodiment, an assay for identifying a cell that expresses a mutant BCR-ABL kinase gene comprises detecting the presence of mutant BCR-ABL mRNA in the cell. Methods for the detection of particular mRNAs in cells are well known and include, for example, hybridization assays using complementary DNA probes (such as in situ hybridization using labeled mutant BCR-ABL kinase riboprobes, Northern blot and related techniques) and various nucleic acid amplification assays (such as RT-PCR using complementary primers specific for a mutant BCR-ABL kinase, and other amplification type detection methods, such as, for example, branched DNA, SISBA, TMA and the like).

The detection methods of the present invention also include methods for identifying amino acid positions within the BCR-ABL polypeptide that may confer at least partial resistance to a tyrosine kinase inhibitor. The methods can comprise the steps of creating a co-crystal of the polypeptide with the BCR-ABL inhibitor, and identifying the amino acid positions of the polypeptide that either contact, bond, interface, or interact with the BCR-ABL inhibitor. Methods of creating crystal structures are known in the art and can include, for example, the use of X-ray crystallography to determine the crystal structure (See, for example, Tokarski et al., Cancer Res (2006), 66(11), 5790-5797) In certain embodiments, the contact or interface amino acids will be at positions 248, 299, 315, and/or 317. In certain embodiments, the contact or interface amino acids will be at positions 244, 248, 255, 290, 299, 313, 315, 316, 317, 318, 320, 321 and/or 380.

The invention encompasses treatment methods based upon the demonstration that patients harboring different BCR-ABL mutations have varying degrees of resistance and/or sensitivity to imatinib and N-(2-chloro-6-methylphenyl)-2-[[6-[4-(2-hydroxyethyl)-1-piperazinyl]-2-methyl-4-pyrimidinyl]amino]-5-thiazolecarboxamide, respectively. Thus the methods of the present invention can be used, for example, in determining whether or not to treat an individual with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; whether or not to treat an individual with a more aggressive dosage regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof; or whether or not to treat an individual with combination therapy, i.e., a combination of tyrosine kinase inhibitors, such as N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and additional BCR-ABL inhibitors(s) (e.g., such as imatinib, AMN107, PD180970, GGP76030, AP23464, SKI 606, NS-187, and/or AZD0530); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a tubulin stabilizing agent (such as, for example, pacitaxol, epothilone, taxane, and the like.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and a famysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and ATP non-competitive inhibitors ONO12380; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and Aurora kinase inhibitor VX-680; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and p38 MAP kinase inhibitor BIRB-796; any other combination disclosed herein.

The terms “treating”, “treatment” and “therapy” as used herein refer to curative therapy, prophylactic therapy, preventative therapy, and mitigating disease therapy. ABL polynucleotide is associated with resistance to inhibition of BCR-ABL kinase activity by N-(2-chloro-6-methylphenyl)-2-[[6-[4-(2-hydroxyethyl)-1-piperazinyl]-2-methyl-4-pyrimidinyl]amino]-5-thiazolecarboxamide, the method comprising determining the sequence of at least one BCR-ABL kinase polynucleotide expressed by the mammalian cell and comparing the sequence of the BCR-ABL kinase polynucleotide to the wild type BCR-ABL kinase polynucleotide sequence. As described herein the polynucleotide identified can encode a polypeptide having at least one amino acid difference from the wild type BCR-ABL kinase amino acid sequence, i.e., a F317A and/or T315I mutation.

In the method disclosed above, the mammalian cell can be a human cancer cell. The human cancer cell can be one obtained from an individual treated having a BCR-ABL associated disorder.

For use herein, a BCR-ABL inhibitor refers to any molecule or compound that can partially inhibit BCR-ABL or mutant BCR-ABL activity or expression. These include inhibitors of the Src family kinases such as BCR/ABL, ABL, c-Src, SRC/ABL, and other forms including, but not limited to, JAK, FAK, FPS, CSK, SYK, and BTK. A series of inhibitors, based on the 2-phenylaminopyrimidine class of pharmacophotes, has been identified that have exceptionally high affinity and specificity for Abl (see, e.g., Zimmerman et al., Bloorg, Med. Chem. Lett. 7, 187 (1997)). All of these inhibitors are encompassed within the term a BCR-ABL inhibitor. Imatinib, one of these inhibitors, also known as STI-571 (formerly referred to as Novartis test compound CGP 57148 and also known as Gleevec), has been successfully tested in clinical trail a therapeutic agent for CML. AMN107, is another BCR-ABL kinase inhibitor that was designed to fit into the ATP-binding site of the BCR-ABL protein with higher affinity than imatinib. In addition to being more potent than imatinib (IC50<30 nM) against wild-type BCR-ABL, AMN107 is also significantly active against 32/33 imatinib-resistant BCR-ABL mutants. In preclinical studies, AMN107 demonstrated activity in vitro and in vivo against wild-type and imatinib-resistant BCR-ABL-expressing cells. In phase I/II clinical trials, AMN107 has produced haematological and cytogenetic responses in CML patients, who either did not initially respond to imatinib or developed imatinib resistance (Weisberg et al., British Journal of Cancer (2006) 94, 1765-1769, incorporated herein by reference in its entirety and for all purposes). SKI-606, NS-187, AZD0530, PD180970, CGP76030, and AP23464 are all examples of kinase inhibitors that can be used in the present invention. SKI-606 is a 4-anilino-3-quinolinecarbonitrile inhibitor of Abl that has demonstrated potent antiproliferative activity leukemia (Green et al., Preclinical Activity of AZD0530, a novel, oral, potent, and selective inhibitor of the Src family kinases. Poster 3161 presented at the EORTC-NCI-AACR, Geneva Switzerland 28 Sep. 2004). PD180970 is a pyrido[2,3-d]pyrimidine derivative that has been shown to inhibit BCR-ABL and induce apoptosis in BCR-ABL expressing leukemic cells (Rosee et al., Cancer Research (2002) 62, 7149-7153). CGP76030 is dual-specific Src and Abl kinase inhibitor shown to inhibit the growth and survival of cells expressing imatinib-resistant BCR-ABL kinases (Warmuth et al., Blood, (2003) 101(2), 664-672). AP23464 is an ATP-based kinase inhibitor that has been shown to inhibit imatinib-resistant BCR-ABL mutants (O'Hare et al., Clin Cancer Res (2005) 11(19), 6987-6993). NS-187 is a selective dual Bcr-Abl/Lyn tyrosine kinase inhibitor that has been shown to inhibit imatinib-resistant BCR-ABL mutants (Kimura et al., Blood, 106(12):3948-3954 (2005)).

A “famysyl transferase inhibitor” can be any compound or molecule that inhibits farnysyl transferase. The farnysyl transferase inhibitor can have formula (II), (R)-2,3,4,5-tetrahydro-1-(1H-imidazol-4-ylmethyl)-3-(phenylmethyl)-4-(2-thienylsulfonyl)-1H-1,4-benzodiazepine-7-carbonitrile, hydrochloride salt. The compound of formula (II) is a cytotoxic FT inhibitor which is known to kill non-proliferating cancer cells preferentially. The compound of formula (II) can further be useful in killing stem cells.

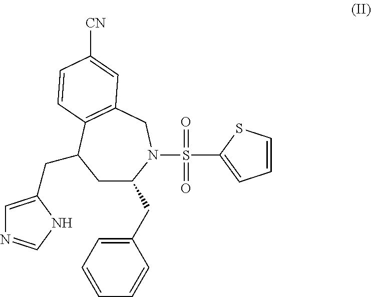

The compound of formula (II), its preparation, and uses thereof are described in U.S. Pat. No. 6,011,029, which is herein incorporated by reference in its entirety and for all purposes. Uses of the compound of formula (II) are also described in WO2004/015130, published Feb. 19, 2004, which is herein incorporated by reference in its entirety and for all purposes. For use herein, combination therapy refers to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof with a second therapy at such time that both the second therapy and N-(2-chloro-6-methylphenyl)-2-Such administration can involve concurrent (i.e., at the same time), prior, or subsequent administration of the second therapy with respect to the administration of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof.

Treatment regimens can be established based upon the presence of one or more mutant BCR-ABL kinases disclosed herein. For example, the invention encompasses screening cells from an individual who may suffer from, or is suffering from, a disorder that is commonly treated with a kinase inhibitor. Such a disorder can include myeloid leukemia or disorders associated therewith, or cancers described herein. The cells of an individual are screened, using methods known in the art, for identification of a mutation in a BCR-ABL kinase. Mutations of interest are those that result in BCR-ABL kinase being constitutively activated. Specific mutations include, for example, F3171 (wherein the phenylalanine at position 317 is replaced with an isoleucine), and T315A (wherein the threonine at position 315 is replaced with an alanine). Other mutations include, for example, E279K, F359C, F359I, L3641, L387M, F486S, D233H, T243S, M244V, G249D, G250E, G251S, Q252H, Y253F, Y253H, E255K, E255V, V256L, Y257F, Y257R, F259S, K262E, D263G, K64R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L364I, E373K, N374D, K378R, V379I, A380T, A380V, D381G, F382L, L387M, M388L, T389S, T392A, T394A, A395G, H396K, H396R, A399G, P402T, T406A, S417Y, F486S or any combination thereof, i.e., M244V, G250E, Q252H, Q252R, Y253F, Y253H, E255K, E255V, T315I, F317L, M351T, E355G, F359V, H396R, F486S and any combination thereof; M244V, E279K, F359C, F359I, L364I, L387M, F486S and any combination thereof; and L248R, Q252H, E255K, V299L, T315I, F317V, F317L, F317S and any combination thereof.

If an activating BCR-ABL kinase mutation is found in the cells from said individual, treatment regimens can be developed appropriately. For example, an identified mutation can indicate that said cells are or will become at least partially resistant to commonly used kinase inhibitors. For example, a F317I or T315A mutation can indicate that the cells in an methyl-4-pyrimidinyl]amino]-5-thiazolecarboxamide. As disclosed herein, in such cases, treatment can include the use of an increased dosing frequency or increased dosage of N-(2-chloro-6-methylphenyl)-2-[[6-[4-(2-hydroxyethyl)-1-piperazinyl]-2-methyl-4-pyrimidinyl]amino]-5-thiazolecarboxamide or a salt, hydrate, or solvate thereof, a combination of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and another kinase inhibitor drug such as imatinib, AMN107, PD180970, GGP76030, AP23464, SKI 606, and/or AZD0530; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; any other combination disclosed herein; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein. In one aspect, an increased level of N-(2-chloro-6-methylphenyl)-2-[[6-[4-(2-hydroxyethyl)-1-piperazinyl]-2-methyl-4-pyrimidinyl]amino]-5-thiazolecarboxamide would be about 10, 20, 30, 40, 50, 60, 70, 80, 90, or 95% more than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual, or about 1.5×, 2×, 2.5×, 3×, 3.5×, 4×, 4.5×, 5×, 6×, 7×, 8×, 9×, or 10× more N-(2-chloro-6-methylphenyl)-2-[[6-[4-(2-hydroxyethyl)-1-piperazinyl]-2-methyl-4-pyrimidinyl]amino]-5-thiazolecarboxamide than the typical N-(2-chloro-6-methylphenyl)-2-[[6-[4-(2-hydroxyethyl)-1-piperazinyl]-2-methyl-4-pyrimidinyl]amino]-5-thiazolecarboxamide dose for a particular indication or for individual.

Additionally, dosage regimens can be further adapted based upon the presence of additional amino acid mutation in a BCR-ABL kinase. As described herein, a mutation in E279K, F359C, F359I, L364I, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, I293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, L364I, E373K, N374D, K378R, V379I, A380T, A380V, D381G, F382L, L387M, M388L, T389S, T392A, T394A, A395G, H396K, H396R, A399G, P402T, T406A, S417Y, F486S, or any combination thereof can indicate that the BCR-ABL kinase has developed at least partial resistance to therapy with a protein kinase inhibitor such as imitinab.

A therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof can be orally administered as an acid salt of N-(2-chloro-6-methylphenyl)-2-[[6-[4-(2-hydroxyethyl)-1-piperazinyl]-2-methyl-4-pyrimidinyl]amino]-5-thiazolecarboxamide. The actual dosage employed can be varied depending upon the requirements of the patient and the severity of the condition being treated. Determination of the proper dosage for a particular situation is within the skill of the art. The effective amount of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof (and Compound I salt) can be determined by one of ordinary skill in the art, and includes exemplary dosage amounts for an adult human of from about 0.05 to about 100 mg/kg of body weight of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof, per day, which can be administered in a single dose or in the form of individual divided doses, such as from 1, 2, 3, or 4 times per day. In certain embodiments, N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof is administered 2 times per day at 70 mg. Alternatively, it can be dosed at, for example, 50, 70, 90, 100, 110, or 120 BID, or 100, 140, or 180 once daily. It will be understood that the specific dose level and frequency of dosing for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, and the like, subject to protein tyrosine kinase-associated disorders. The same also applies to Compound II or any combination of Compound I and II, or any combination disclosed herein.

chloro-6-methylphenyl)-2-[[6-[4-(2-hydroxyethyl)-1-piperazinyl]-2-methyl-4-pyrimidinyl]amino]-5-thiazolecarboxamide and imatinib, is disclosed herein. For example, an individual can be determined to be a positive responder (or cells from said individual would be expected to have a degree of sensitivity) to a certain kinase inhibitor based upon the presence of a mutant BCR-ABL kinase. As disclosed herein, cells that exhibit certain mutations at amino acid positions 315 and 317 of BCR-ABL kinase, can develop at least partial resistance to of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof. Therefore, individuals suffering from a protein tyrosine kinase-associated disorder whose cells exhibit such a mutation are or would be expected to be partially negative responders to a particular treatment regimen with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof but a positive responder to a more aggressive treatment regimen of N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof or to combination therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and imatinib or other therapy.

A treatment regimen is a course of therapy administered to an individual suffering from a protein kinase associated disorder that can include treatment with one or more kinase inhibitors, as well as other therapies such as radiation and/or other agents (i.e., combination therapy). When more than one therapy is administered, the therapies can be administered concurrently or consecutively (for example, more than one kinase inhibitor can be administered together or at different times, on a different schedule). Administration of more than one therapy can be at different times (i.e., consecutively) and still be part of the same treatment regimen. As disclosed herein, for example, cells from an individual suffering from a protein kinase associated disorder can be found to develop at least partial resistance to N-(2-chloro-6-methylphenyl)-2-[[6-[4-(2-hydroxyethyl)-1-piperazinyl]-2-methyl-4-pyrimidinyl]amino]-5-thiazolecarboxamide. Based upon the present discovery that such cells can be sensitive to combination therapy or a more aggressive dosage or dosing regimen of N-(2-chloro-6-methylphenyl)-2-[[6-[4-(2-hydroxyethyl)-1-piperazinyl]-2-methyl-4-pyrimidinyl]amino]-5-monotherapy, or in combination with another kinase inhibitor, or in combination with another agent as disclosed herein. Additionally, the combination can be administered with radiation or other known treatments.

Therefore the present invention includes a method for establishing a treatment regimen for an individual suffering from a protein tyrosine kinase associated disorder or treating an individual suffering from a protein tyrosine kinase disorder comprising determining whether a biological sample obtained from an individual has at least a F317 I and/or T315A mutation in the BCR-ABL kinase, and administering to the subject an appropriate treatment regimen based on whether the mutation is present. The determination can be made by any method known in the art, for example, by screening said sample of cells for the presence of at least one mutation in a BCR-ABL kinase sequence or by obtaining information from a secondary source that the individual has the specified BCR-ABL kinase mutation.

In practicing the many aspects of the invention herein, biological samples can be selected from many sources such as tissue biopsy (including cell sample or cells cultured therefrom; biopsy of bone marrow or solid tissue, for example cells from a solid tumor), blood, blood cells (red blood cells or white blood cells), serum, plasma, lymph, ascetic fluid, cystic fluid, urine, sputum, stool, saliva, bronchial aspirate, CSF or hair. Cells from a sample can be used, or a lysate of a cell sample can be used. In certain embodiments, the biological sample is a tissue biopsy cell sample or cells cultured therefrom, for example, cells removed from a solid tumor or a lysate of the cell sample. In certain embodiments, the biological sample comprises blood cells.

Pharmaceutical compositions for use in the present invention can include compositions comprising one or a combination of inhibitors of a mutant BCR-ABL kinase in an effective amount to achieve the intended purpose. The determination of an effective dose of a pharmaceutical composition of the invention is well within the capability of those skilled in the art. A therapeutically effective dose refers to that amount of active ingredient which ameliorates the symptoms or condition. Therapeutic efficacy and toxicity can be determined by standard pharmaceutical procedures in cell cultures or experimental animals, for example the ED50 (the dose therapeutically effective in 50% of the population) and LD50 (the dose lethal to 50% of the population).

Dosage regimens involving N-(2-chloro-6-methylphenyl)-2-[[6-[4-(2-hydroxyethyl)-1-piperazinyl]-2-methyl-4-pyrimidinyl]amino]-5-thiazolecarboxamide useful in “Hematologic and Cytogenetic Responses in imatinib-Resistant Accelerated and Blast Phase Chronic Myeloid Leukemia (CML) Patients Treated with the Dual SRC/ABL Kinase Inhibitor N-(2-chloro-6-methylphenyl)-2-[[6-[4-(2-hydroxyethyl)-1-piperazinyl]-2-methyl-4-pyrimidinyl]amino]-5-thiazolecarboxamide: Results from a Phase I Dose Escalation Study.”, by Moshe Talpaz, et al; which are hereby incorporated herein by reference in their entirety and for all purposes.

A “therapeutically effective amount” of an inhibitor of a mutant BCR-ABL kinase can be a function of the mutation present. For example Shah et al disclose that cell lines with certain mutations in BCR-ABL kinase are more sensitive to N-(2-chloro-6-methylphenyl)-2-[[6-[4-(2-hydroxyethyl)-1-piperazinyl]-2-methyl-4-pyrimidinyl]amino]-5-thiazolecarboxamide than cell lines with different BCR-ABL kinase mutations. As disclosed therein, cells comprising a F317L mutation in BCR-ABL kinase requires three to five-fold higher concentration of N-(2-chloro-6-methylphenyl)-2-[[6-[4-(2-hydroxyethyl)-1-piperazinyl]-2-methyl-4-pyrimidinyl]amino]-5-thiazolecarboxamide than cell lines expressing a Q252R mutation. One skilled in the art will appreciate the difference in sensitivity of the mutant BCR-ABL kinase cells and determine a therapeutically effective dose accordingly.

Examples of predicted therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide that may be warranted based upon the relative sensitivity of BCR-ABL kinase mutants to N-(2-chloro-6-methylphenyl)-2-[[6-[4-(2-hydroxyethyl)-1-piperazinyl]-2-methyl-4-pyrimidinyl]amino]-5-thiazolecarboxamide compared to wild-type BCR-ABL kinase can be determined using various in vitro biochemical assays including cellular proliferation, BCR-ABL tyrosine phosphorylation, peptide substrate phosphorylation, and/or autophosphorylation assays. For example, approximate therapeutically effective doses of N-(2-chloro-6-methylphenyl)-2-[[6-[4-(2-hydroxyethyl)-1-piperazinyl]-2-methyl-4-pyrimidinyl]amino]-5-thiazolecarboxamide can be calculated based upon multiplying the typical dose with the fold change in sensitivity in anyone or more of these assays for each BCR-ABL kinase mutant. O'Hare et al. (Cancer Research, 65(11):4500-5 (2005), which is hereby incorporated by reference in its entirety and for all purposes) performed analysis of the relative sensitivity of N-(2-chloro-6-methylphenyl)-2-[[6-[4-(2-hydroxyethyl)-1-piperazinyl]-2-methyl-4-pyrimidinyl]amino]-5-thiazolecarboxamide with several clinically relevant BCR-ABL Kinase mutants. For example, the E255V mutant had a fold change of “1” in the GST-Abl kinase assay, piperazinyl]-2-methyl-4-pyrimidinyl]amino]-5-thiazolecarboxamide for patients harboring this mutation could range, for example, anywhere from 1 to 14 fold higher than the typical dose. Accordingly, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide for any of the BCR-ABL kinase mutants can be, for example, 1, 2, 3, 4, 5, 6, 7, 8, 9, 10, 11, 12, 13, 14, 15, 16, 17, 18, 19, 20, 25, 30, 35, 40, 50, 60, 70, 80, 90, 100, 125, 150, 175, 200, 225, 250, or 300 folder higher than the prescribed dose. Alternatively, therapeutically relevant doses of N-(2-chloro-6-methylphenyl)-2-[[6-[4-(2-hydroxyethyl)-1-piperazinyl]-2-methyl-4-pyrimidinyl]amino]-5-thiazolecarboxamide can be, for example, 0.9×, 0.8×, 0.7×, 0.6×, 0.5×, 0.4×, 0.3×, 0.2×, 0.1×, 0.09×, 0.08×, 0.07×, 0.06×, 0.05×, 0.04×, 0.03×, 0.02×, or 0.01× of the prescribed dose.

According to O'hare et al., the M244V mutant had a fold change of “1.3” in the GST-Abl kinase assay, a fold change of “1.1” in the autophosphorylation assay, and a fold change of “2” in the cellular proliferation assay; the G250E mutant had a fold change of “0.5” in the GST-Abl kinase assay, a fold change of “3” in the autophosphorylation assay, and a fold change of “2” in the cellular proliferation assay; the Q252H mutant had a fold change of “4” in the cellular proliferation assay; the Y253F mutant had a fold change of “0.6” in the GST-Abl kinase assay, a fold change of “4” in the autophosphorylation assay, and a fold change of “4” in the cellular proliferation assay; the Y253H mutant had a fold change of “3” in the GST-Abl kinase assay, a fold change of “2” in the autophosphorylation assay, and a fold change of “2” in the cellular proliferation assay; the E255K mutant had a fold change of “0.3” in the GST-Abl kinase assay, a fold change of “2” in the autophosphorylation assay, and a fold change of “7” in the cellular proliferation assay; the F317L mutant had a fold change of “1.5” in the GST-Abl kinase assay, a fold change of “1.4” in the autophosphorylation assay, and a fold change of “9” in the cellular proliferation assay; the M351T mutant had a fold change of “0.2” in the GST-Abl kinase assay, a fold change of “2” in the autophosphorylation assay, and a fold change of “1.4” in the cellular proliferation assay; the F359V mutant had a fold change of “0.8” in the GST-Abl kinase assay, a fold change of “2” in the autophosphorylation assay, and a fold change of “3” in the cellular proliferation assay; the H396R mutant had a fold change of “1.3” in the GST-Abl kinase assay, a fold change of “3” in the autophosphorylation assay, and a fold change of “2” in the cellular proliferation assay.

pyrimidinyl]amino]-5-thiazolecarboxamide, or combinations of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide and a farnysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide and another protein tyrosine kinase inhibitor; any other combination discloses herein; an increased dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide; and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide disclosed herein, may be warranted. Alternatively, combinations of N-(2-chloro-6-methylphenyl)-2-[[6-[4-(2-hydroxyethyl)-1-piperazinyl]-2-methyl-4-pyrimidinyl]amino]-5-thiazolecarboxamide with a T315I inhibitor may also be warranted.

Therefore, the present invention provides methods of treating an individual suffering from a protein tyrosine kinase-associated disorder such as a BCR-ABL associated disorder, for example, a BCR-ABL-associated cancer, (where such individual is naïve to treatment with a kinase inhibitor (i.e., has not previously been treated with such) or has been treated with one or more kinase inhibitors (for example, has been treated with imatinib or N-(2-chloro-6-methylphenyl)-2-[[6-[4-(2-hydroxyethyl)-1-piperazinyl]-2-methyl-4-pyrimidinyl]amino]-5-thiazolecarboxamide)), comprising determining whether the individual has a mutant BCR-ABL that renders the individual less sensitive to therapy with N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, hydrate, or solvate thereof and, based on whether the mutant kinase is present, administering to said individual N-(2-chloro-6-methylphenyl)-2-[[6-[4-(2-hydroxyethyl)-1-piperazinyl]-2-methyl-4-pyrimidinyl]amino]-5-thiazolecarboxamide, preferably as an active agent in a pharmaceutical composition at a dose and/or frequency of dosing selected based on said assay (e.g., based on the sensitivity of such mutant(s) relative to with wild-type BCR-ABL kinase), and/or in combination with another protein tyrosine kinase inhibitor, including, for example, imatinib, AMN107, PD180970, suitable for the treatment of said protein tyrosine kinase-associated disorder disclosed herein, said other kinase inhibitor and/or other agent being administered simultaneously or sequentially with the administration of N-(2-chloro-6-methylphenyl)-2-[[6-[4-(2-hydroxyethyl)-1-piperazinyl]-2-methyl-4-pyrimidinyl]amino]-5-thiazolecarboxamide.

According to the present invention, dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. See, e.g., the latest Remington's (Remington's Pharmaceutical Science, Mack Publishing Company, Easton, Pa.)

### VI. Identification of Mutant BCR-ABL Interactors and Therapeutic Compounds

The present invention provides methods of identifying compounds that interact with, i.e., specifically bind to, a mutant BCR-ABL polypeptide. The compound can be a protein, small molecule, or other agent that can interact with the mutant BCR-ABL polypeptide. Methods of determining whether a test compound can interact with a specific polypeptide are known in the art.

For example, in certain embodiments, the present invention comprises a method of screening for a molecule that interacts with an mutant BCR-ABL amino acid sequence comprising the steps of contacting a population of molecules with the BCR-ABL amino acid sequence, allowing the population of molecules and the BCR-ABL amino acid sequence to interact under conditions that facilitate an interaction, determining the presence of a molecule that interacts with the BCR-ABL amino acid sequence, and then separating molecules that do not ABL amino acid sequence. The identified molecule can be used to modulate a function performed by BCR-ABL.

Screening chemical libraries for molecules which modulate, e.g., inhibit, antagonize, or agonize or mimic, are known in the art. The chemical libraries, for example, can be peptide libraries, peptidomimetic libraries, chemically synthesized libraries, recombinant, e.g., phage display libraries, and in vitro translation-based libraries, other non-peptide synthetic organic libraries (e.g. libraries of 2-phenylaminopyrimidines, quinazolines or pyrazolo-pyrrolo-pyridopyrimidi-nes and the like).

Exemplary libraries are commercially available from several sources (ArQule, Tripos/PanLabs, ChemDesign, Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position can have from 1-20 chemicals that can be screened by administration to a well containing the interactions of interest. Thus, if modulation is detected, smaller and smaller pools of interacting pairs can be assayed for the modulation activity. By such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome-based libraries are exemplary types of libraries that can be used.

In certain embodiments, one can screen peptide libraries to identify molecules that interact with the mutant BCR-ABL protein sequences. In such methods, peptides that bind to a molecule such as mutant BCR-ABL can be identified by screening libraries that encode a random or controlled collection of amino acids. Peptides encoded by the libraries can be expressed as fusion proteins of bacteriophage coat proteins, the bacteriophage particles can then be screened against the protein of interest.

Accordingly, peptides having a wide variety of uses, such as therapeutic, prognostic or diagnostic reagents, are thus identified without any prior information on the structure of the expected ligand or receptor molecule. Typical peptide libraries and screening methods that can be used to identify molecules that interact with mutant BCR-ABL protein their entirety.

Small molecules and ligands that interact with mutant BCR-ABL can be identified through related embodiments of such screening assays. For example, small molecules can be identified that interfere with protein function, including molecules that interfere with a mutant BCR-ABL's ability to mediate phosphorylation and de-phosphorylation.

In certain embodiments, a method of identifying a compound which specifically binds a mutant BCR-ABL as provided herein comprises the steps of: contacting said mutant BCR-ABL with a test compound under conditions favorable to binding; and then determining whether said test compound binds to said mutant BCR-ABL so that a compound which binds to said mutant BCR-ABL can be identified. As the interaction between various Abelson tyrosine kinases and a variety of test compounds have been previously described, skilled artisans are familiar with the conditions conducive to binding.

In certain embodiments, cells will be transfected with a construct encoding the mutant BCR-ABL, contacted with a test compound that is tagged or labelled with a detectable marker and analyzed for the presence bound test compound. In contexts where the transfected cells are observed to preferentially bind the test compound as compared to cells that have not been transfected with a mutant BCR-ABL ARS construct, this indicates that the test compounds is binding to the mutant BCR-ABL protein expressed by those cells. The binding of the compound is typically determined by any one of a wide variety of assays known in the art such as ELISA, RIA, and/or BIAcore assays.

A test compound which binds to a mutant BCR-ABL can be further screened for the inhibition of a biological activity (e.g. tyrosine kinase activity) of said mutant BCR-ABL. Such an embodiment includes, for example determining whether said test compound inhibits the tyrosine kinase activity of the mutant BCR-ABL by utilizing molecular biological protocols to create recombinant contracts whose enzymological and biological properties can be examined directly. A specific biological activity such as resistance to a protein kinase inhibitor can be measured using standard kinase assays and transformation assays. Enzymology is performed for example, by measuring tyrosine kinase activity in vitro or in mutant BCR-ABL expressing cells using standard assays. Such methods typically comprise the steps of examining the kinase activity or growth potential of a BCR-ABL mutant expressing cell line in the absence of a test compound and comparing this to the kinase activity or growth potential of a BCR-ABL mutant expressing cell line in the presence of a test compound, wherein an decrease in the kinase BCR-ABL mutant. For example, BCR-ABL kinase activity can be measured by monitoring the phosphotyrosine content of Crkl using methods known in the art and described in the examples section herein.

Alternative methods for measuring the enzymological and biological property of BCR-ABL mutants variety of assays for measuring the enzymological properties of protein kinases such as Abl are known in the art, and include, for example, those described in Konopka et al., Mol Cell Biol. November 1985; 5(11):3116-23; Davis et al., Mol Cell Biol. January 1985; 5(1):204-13; and Konopka et al., Cell. Jul. 1, 1984; 37(3):1035-42 the contents of which are incorporated herein by reference in their entirety and for all purposes. Using such assays the skilled artisan can measure the enzymological properties of mutant BCR-ABL protein kinases.

A variety of bioassays for measuring the transforming activities of protein kinases such as Abl are known in the art, for example those described in Lugo et al., Science. Mar. 2, 1990; 247(4946):1079-82; Lugo et al., Mol Cell Biol. March 1989; 9(3):1263-70; Klucher et al., Blood. May 15, 1998; 91(10):3927-34; Renshaw et al., Mol Cell Biol. March 1995; 15(3):1286-93; Sitard et al., Blood. Mar. 15, 1994; 83(6):1575-85; Laneuville et al., Cancer Res. Mar. 1, 1994; 54(5):1360-6; Laneuville et al., Blood. Oct. 1, 1992; 80(7):1788-97; Mandanas et al., Leukemia. August 1992; 6(8):796-800; and Laneuville et al., Oncogene. February 1991; 6(2):275-82 the contents of which are incorporated herein by reference in their entirety for all purposes. Using such assays the skilled artisan can measure the phenotype of mutant BCR-ABL protein kinases.

### VII. Kits

For use in the diagnostic and therapeutic applications described or suggested above, kits are also provided by the invention. Such kits can, for example, comprise a carrier means being compartmentalized to receive in close confinement one or more container means such as vials, tubes, and the like, each of the container means comprising one of the separate elements to be used in the method. For example, one of the container means can comprise a probe that is or can be detectably labeled. Such probe can be an antibody or polynucleotide specific for a mutant BCR-ABL kinase protein or a mutant BCR-ABL kinase gene or message, respectively. Where the kit utilizes nucleic acid hybridization to detect the target nucleic acid, the kit can also have containers containing nucleotide(s) for amplification of the target nucleic acid sequence and/or a container comprising a reporter-means, such as a biotin-binding protein,

The kit of the invention will typically comprise the container described above and one or more other containers comprising materials desirable from a commercial and user standpoint, including buffers, diluents, filters, needles, syringes, and package inserts with instructions for use. A label can be present on the container to indicate that the composition is used for a specific therapy or non-therapeutic application, and can also indicate directions for either in vivo or in vitro use, such as those described above.

Kits useful in practicing therapeutic methods disclosed herein can also contain a compound that is capable of inhibiting a mutant BCR-ABL kinase. Specifically contemplated by the invention is a kit comprising a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a tubulin stabilizing agent (e.g., pacitaxol, epothilone, taxane, etc.); a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and a famysyl transferase inhibitor; a combination of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, and another protein tyrosine kinase inhibitor, such as, imatinib, AMN107, PD180970, GGP76030, AP23464, SKI 606, NS-187, and/or AZD0530; an increased dose and/or dosing frequency regimen of N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, relative a treatment regimen suitable for such other forms of such BCR-ABL kinase (e.g., wild-type); and any other combination or dosing regimen comprising N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof disclosed herein, useful in treating mammals suffering from a BCR-ABL associated disorder, including mutant BCR-ABL associated disorder. For example, kits useful in identifying a mutant BCR-ABL kinase in a mammalian patient (e.g., a human) suffering from a cancer that is completely or partially resistant to, or has developed complete or partial resistance to, N-(2-chloro-6-methylphenyl)-2-[[6-[4-(2-hydroxyethyl)-1-piperazinyl]-2-methyl-4-pyrimidinyl]amino]-5-thiazolecarboxamide or salt, hydrate, or solvate thereof, imatinib, or another protein tyrosine kinase inhibitor and where said kits also comprise a therapeutically effective amount of the combination or increased dose or dosing regimen, are contemplated herein.

typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips, and the like), optical media (e.g., CD ROM), and the like. Such media can include addresses to internet sites that provide such instructional materials.

The kit can also comprise, for example, a means for obtaining a biological sample from an individual. Means for obtaining biological samples from individuals are well known in the art, e.g., catheters, syringes, and the like, and are not discussed herein in detail.

The following Exemplary Embodiments of specific aspects for carrying out the present invention are offered for illustrative purposes only, and are not intended to limit the scope of the present invention in any way.

### EXEMPLARY EMBODIMENTS

### Example 1

### Exemplary Methods for Detecting BCR-ABL Kinase Mutations

N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are two potent BCR-ABL kinase inhibitors that are effective in treating CML and solid tumors. Provided herein are exemplary combination therapies and dosing regimens that will be useful in treating cancers which are resistant to protein tyrosine kinase inhibitor agents, such as imatinib and other kinase inhibitors, and specifically including cancers involving one or more mutations in BCR-ABL kinase.

A significant aspect of this combination therapy is the detection of the mutations in BCR-ABL kinase. If a mutant BCR-ABL kinase of the present invention is present in a patient, it indicates an individual can be selected for combination therapy, or more aggressive dosing regimens (e.g., higher and/or more frequent doses), or a combination of aggressive dosing regimen and combination therapy. Furthermore, if a specific BCR-ABL kinase mutant is detected, the amount of either or both inhibitors can be increased or decreased in order to enhance the therapeutic effect of the regimen.

There are several methods that can be used to detect a mutant BCR-ABL kinase in cancer patients, particularly CML patients. They include methods for detecting BCR-ABL kinase polynucleotides and BCR-ABL kinase proteins, as well as methods for identifying cells that express BCR-ABL kinase. Detection of certain mutant BCR-ABL kinasse in a patient would be diagnostic that such patients either are or will become at least partially resistant to imatinib therapy or N-(2-chloro-6-methylphenyl)-2-[[6-[4-(2-hydroxyethyl)-1-piperazinyl]-2-methyl-4-pyrimidinyl]amino]-5-thiazolecarboxamide therapy. As discussed in detail below, the status of BCR-ABL kinase gene products in patient samples can be analyzed by a variety of protocols well known in the art including, for example, immunohistochemical analysis, the variety of Northern blotting techniques including in situ hybridization, RT-PCR analysis (for example on laser capture micro-dissected samples), western blot analysis, tissue array analysis, microarray analysis, genotyping methods, and mass-spectroscopic methods.

Methods of identifying the nucleic acid and the amino acid of a mutant BCR-ABL kinase are known in the art.

10 independent clones in both directions. This strategy allows one to quantify fluctuations in different clones from the same patient over time. Typical methodologies are for such protocols are provided below.

Blood samples can be obtained from patients enrolled in clinical trials in the treatment of CML. RNA is then extracted using TriAgent or TriAzol. cDNA synthesis is performed using MMTV reverse transcriptase. Polymerase chain reaction (PCR) is performed to amplify the cDNA, using primers CM10 (5′-GAAGCTTCTCCCTGACATCCGT-3′) (SEQ ID NO: 3) and 3′ Abl KD (5′-GCCAGGCTCTCGGGTGCAGTCC-3′) (SEQ ID NO:4). A second round of PCR is performed to isolate the kinase domain using primers 5′ Abl KD, (5′-GCGCAACAAGCCCACTGTCTATGG-3′) (SEQ ID NO: 5) and 3′ Abl KD. The resultant 0.6 Kb fragment is then ligated into pBluescript II KS+digested with Eco RV. Bacterial transformants are plated on media containing ampicillin and X-gal. Ten white colonies per cDNA are inoculated into media and miniprep DNA is isolated. Sequencing of each clone is then performed using M13 universal forward (CGCCAGGGTTTTCCCAGTCACGAC; SEQ ID NO:6) and M13 reverse (AGCGGATAACAATTTCACACAGGA; SEQ ID NO:7) primers. Because two rounds of amplification will be employed, a mutation was considered present if it was detected on both strands of at least two independent clones per patient.

Alternatively, antibodies that immunospecifically bind to mutant BCR-ABL kinase can be used to detect the presence of a mutant BCR-ABL kinase in a sample. First, mutant BCR-ABL kinase can be generated by site directed mutagenesis. Cell lines expressing these mutant BCR-ABL kinase isoforms will then be created. Next, antibodies against mutant BCR-ABL kinase isoforms will be produced. Expression of BCR-ABL kinase and its mutant isoforms will be documented by Western blot analysis.

Specifically, site directed mutagenesis can be used to create the BCR-ABL kinase mutations (QuickChange Kit, Stratagene, La Jolla, Calif.) and all mutations will be confirmed by bidirectional sequencing (O'Farrell, A. M., et al., Blood, 101:3597-3605 (2003)). Retroviral transduction is performed and Ba/F3 cell lines stably expressing mutant BCR-ABL kinase isoforms are generated by double selection for G418 resistance and IL-3 independent growth (Yee, K. W., et al., Blood, 100:2941-2949 (2002); Yee, K. W., et al., Blood, 104:4202-4209 (2004); Tse, K. F., et al., Leukemia, 14:1766-1776 (2000); Schittenhelm, M. M., et al., manuscript submitted (2005)). Transient transfections of CHO-K1 chinese hamster cell lines with BCR-ABL kinase wild type (“WT”) or mutant isoforms are performed using a lipofection-pyrimidinyl]amino]-5-thiazolecarboxamide 24 hours after transfection (Heinrich, M. C., et al., Journal of Clinical Oncology, 21:4342-4349 (2003)). Alternatively, cells can be treated with any of the combinations outlined herein, or using increased levels of N-(2-chloro-6-methylphenyl)-2-[[6-[4-(2-hydroxyethyl)-1-piperazinyl]-2-methyl-4-pyrimidinyl]amino]-5-thiazolecarboxamide.

An anti-BCR-ABL kinase rabbit polyclonal antibody, an anti-STAT3 mouse monoclonal antibody (both Santa Cruz Biotechnology, Santa Cruz, Calif.), an anti-AKT (polyclonal) rabbit antibody (Cell Signaling Technology, Beverly Mass.) and an anti-MAP kinase 1/2 (Erk 1/2) rabbit monoclonal antibody (Upstate Biotechnology, Lake Placid, N.Y.) can be used at a 1:5000 to 1:2000 dilution. Anti-phosphotyrosine BCR-ABL antibodies (Tyr568/570 and Tyr703), an anti-phosphothreonine/tyrosine MAP kinase (Thr202/Tyr204) antibody, an anti-phosphothreonine (Thr308) and an anti-phosphoserine (Ser473) AKT antibody, an anti-phosphotyrosine (Tyr705) STAT3 antibody and an unspecific anti-phosphotyrosine antibody (clone pY20) are used at dilutions of 1:100 to 1:2000 (all Cell Signaling Technology, Beverly Mass.). Peroxidase conjugated goat anti-mouse antibody and goat anti-rabbit antibody will be used at 1:5000 and 1:10,000 dilutions respectively (BioRad; Hercules, Calif.). Protein A/G PLUS-Agarose immunoprecipitation reagent shall be purchased from Santa Cruz Biotechnology (Santa Cruz, Calif.). imatinib, N-(2-chloro-6-methylphenyl)-2-[[6-[4-(2-hydroxyethyl)-1-piperazinyl]-2-methyl-4-pyrimidinyl]amino]-5-thiazolecarboxamide, paclia tubulin stabilizing agent (e.g., pacitaxol, epothilone, taxane, etc.), and another agents useful in combination with N-(2-chloro-6-methylphenyl)-2-[[6-[4-(2-hydroxyethyl)-1-piperazinyl]-2-methyl-4-pyrimidinyl]amino]-5-thiazolecarboxamide, are dissolved in DMSO to create 10 mM stock solutions and be stored at −20° C.

Western blot assays can be conducted as follows. ˜5×10⁷ cells are exposed to varying concentrations of N-(2-chloro-6-methylphenyl)-2-[[6-[4-(2-hydroxyethyl)-1-piperazinyl]-2-methyl-4-pyrimidinyl]amino]-5-thiazolecarboxamide and cultured for 90 minutes at 37° C. in a 5% CO₂ atmosphere. Cell pellets are lysed with 100-150 μL of protein lysis buffer (50 mM Tris, 150 mM NaCl, 1% NP-40, 0.25% deoxycholate with added inhibitors aprotinin, AEBSF, leupeptin, pepstatin, sodium orthovanadate, and sodium pyruvate). 500-2000 microgram of protein from cell lysates are used for immunoprecipitation experiments and 75-200 microgram of protein from cell lysates are used for whole cell protein analysis by western immunoblot assays as previously described in Hoatlin, M. E., et al., Blood, 91:1418-1425 (1998).

corresponding to the ATP binding pocket and the activation loop domain of BCR-ABL is critical to the selectivity of imatinib and is the region known to harbor the most imatinib-resistant and protein tyrosine kinase inhibitor mutations. Sequencing of this region can most efficiently reveal the patients' CML clinical profile, and hence the appropriate combination therapy and/or dosing regimen. Briefly, RNA is extracted from purified peripheral blood or bone marrow cells with TriReagent-LS (Molecular Research Center, Inc., Cincinnati, Ohio). Total RNA is subjected to RT-PCR by using the same protocol and primers as described supra. PCR products are cloned into the pCR2.1 TA cloning vector (Invitrogen, Carlsbad, Calif.). Both strands of a 579-bp region are sequenced with the 5′ ABL primer and M13 forward primer or M13 forward and reverse primer set for the 1327-bp and the 579-bp fragments, respectively, on an ABI prism 377 automated DNA sequencer (PE Applied Biosystems, Foster City, Calif.). Sequence analysis is then performed using the ClustalW alignment algorithm). Any detected mutation is then confirmed by analysis of genomic DNA. Briefly, genomic DNA is extracted from purified bone marrow or peripheral blood cells with the QiaAMP Blood Mini Kit (Qiagen, Inc., Valencia, Calif.). A 361-bp DNA fragment is amplified by PCR with two primers (5′-GCAGAGTCAGAATCCTTCAG-3′ (SEQ ID NO: 8) and 5′-TTTGTAAAAGGCTGCCCGGC-3′) (SEQ ID NO: 9) which are specific for intron sequences 5′ and 3′ of ABL exon 3, respectively. PCR products are cloned and sequenced.

Additional methods of detecting mutant BCR-ABL kinases is disclosed in O'Hare et al. (Cancer Research, 65(11):4500-5 (2005), which is hereby incorporated by reference in its entirety).

### Example 2

### Exemplary Method of Assessing the Potential of the Combination Therapy

The combination of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib can be studied in mouse models of imatinib-resistant or protein tyrosine kinase inhibitor resistant, BCR-ABL-dependent disease. A series of such pharmacodynamic experiments will help to determine the optimal dosing regimen for different mutant BCR-ABL isoforms in vivo. Pharmacodynamic experiments are well known in the art and one skilled in the art would readily appreciate that such experiments can be modified to alter existing conditions, as applicable. Briefly, severe combined immuno-deficient mice are injected intravenously with Ba/F3 cells expressing different BCR-ABL wild-type or mutant isoforms as well as the firefly luciferase gene. typically resulting in death. To assess the ability of combination therapy, or a modified dosing regimen, to inhibit BCR-ABL in vivo, BCR-ABL kinase activity in splenocyte lysates prepared at various time points after administration of a different single dose of 0, 0.5, 1, 5, and 10 micromoles per liter of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination by oral gavage will be assessed. Phosphorylation of the adapter protein CRKL, a known BCR-ABL substrate (T. Oda et al., J. Biol. Chem. 269, 22925 (1994)), will be monitored to gauge the efficacy of the combination therapy. On the basis of a series of such pharmacodynamic experiments, an proper dose of the combination will be chosen for efficacy studies. Then, mice will be documented by bioluminescence imaging before and after dosing. On the basis of a series of such pharmacodynamic experiments, the optimal dosing regimen and/or combination therapy can be identified. Mice are dosed with combination or vehicle alone by gavage for 2 weeks, beginning 3 days after injection of Ba/F3 cells, and disease burden is then assessed by bioluminescence imaging. All vehicle-treated mice are expected to develop progressive disease. In contrast, combination-treated mice harboring nonmutant BCR-ABL or the clinically common imatinib-resistant and protein tyrosine kinase inhibitor resistant mutations described herein are expected to develop less or no progressive disease. It is also expected that different optimal dosing regimens will be identified for different BCR-ABL isoforms. Such dosing difference can be taken into consideration in the treatment of patients with a known BCR-ABL mutation(s).

### Example 3

### Exemplary Method of Assessing the Safety and Efficacy of Protein Tyrosine Kinase Combination Therapy and/or Modified Dosing Regimens

Previous findings have shown that both N-(2-chloro-6-methylphenyl)-2-[[6-[4-(2-hydroxyethyl)-1-piperazinyl]-2-methyl-4-pyrimidinyl]amino]-5-thiazolecarboxamide and imatinib are highly selective for leukemic versus normal hematopoietic cells (B. J. Druker et al., Nature Med. 2. 561 (1996) and N. P. Shah et al., Science 305. 399 (2004)). Such high selectivity demonstrates the high safety and efficacy of these inhibitors, and the expected efficacy of their combination. To assess the efficacy of the combination on human bone marrow progenitors, the compounds are tested in vitro in colony-forming-unit (CFU) assays. A series of concentrations of N-(2-chloro-6-methylphenyl)-2-[[6-[4-(2-hydroxyethyl)-1-piperazinyl]-2-methyl-4-pyrimidinyl]amino]-5-thiazolecarboxamide and imatinib combination, or other combinations disclosed herein, are applied to bone marrow progenitors isolated from healthy granulocyte-monocyte (GM) colonies from CML patient marrow samples will be analyzed by polymerase chain reaction (PCR) analysis in order to detect the sensitivity of selection for growth of rare normal progenitors present in these leukemic marrow samples. Briefly, bone marrow is harvested from clinical subjects. Viable frozen Ficoll-Hypaque-purified mononuclear cells are thawed and grown overnight in Iscove's Media supplemented with 10% Fetal calf serum, 1-glutamine, pen-strep, and stem cell factor (100 ug/ml) at a density of 5×10⁵/ml. After 24 hours, viable cells are quantitated and plated in Methocult media (Cell Signal Technologies, Beverly, Mass.) at 1×10⁴ and 1×10⁵ cells per plate in the presence of 5 nM N-(2-chloro-6-methylphenyl)-2-[[6-[4-(2-hydroxyethyl)-1-piperazinyl]-2-methyl-4-pyrimidinyl]amino]-5-thiazolecarboxamide or vehicle. Experiments are performed in triplicate. On day 11, erythroid blast-forming unit (BFU-E) and granulocyte-macrophage colony forming units (CFU-GM) will be quantitated. On day 14, colonies will be isolated with a pipet tip, and RNA will be isolated using a Qiagen Rneasy kit. A primer complementary to the region of ABL approximately 200 nucleotides downstream of the BCR-ABL mRNA (5′-CGGCATTGCGGGACACAGGCCCATGGTACC; SEQ ID NO:10) junction is annealed to purified RNA. cDNA is synthesized using mouse Moloney leukemia virus (MMLV) reverse transcriptase, and subjecting to 40 cycles of PRC using either a BCR (5′-TGACCAACTCGTGTGTGAAACT; SEQ ID NO:11) or ABL type Ia 5′ primer (GGGGAATTCGCCACCATGTTGGAGATCTGCCTGA; SEQ ID NO:12) as a control for the quality of RNA.

### Example 4

### Exemplary Methods for Measuring of BCR-ABL Kinase Activity Via the Phosphotyrosine Content of Crkl

The ability of a combination therapy or more aggressive dosing regimen of the present invention to effectively overcome N-(2-chloro-6-methylphenyl)-2-[[6-[4-(2-hydroxyethyl)-1-piperazinyl]-2-methyl-4-pyrimidinyl]amino]-5-thiazolecarboxamide or imatinib resistance, to inhibit BCR-ABL activity, or to inhibit BCR-ABL mutant activity, the phosphotyrosine content of Crkl, an adaptor protein which is specifically and constitutively phosphorylated by BCR-ABL in CML cells can be used (see, e.g. J. ten Hoeve et al., Blood 84, 1731 (1994); T. Oda et al., J. Biol. Chem. 269, 22925 (1994); and G. L. Nichols et al., Blood 84, 2912 (1994)). The phosphotyrosine content of Crkl has been shown to be reproducibly and quantitatively measured in clinical specimens. Crkl binds BCR-ABL directly and plays a phosphorylated, Crkl migrates with altered mobility in SDS-PAGE gels and can be quantified using densitometry. Sawyers et al (U.S. Ser. No. 10/171,889, filed Jun. 16, 2002; incorporated herein by reference) have shown that Crkl phosphorylation in primary CML patient cells was inhibited in a dose-dependent manner when exposed to STI-571 and correlated with dephosphorylation of BCR-ABL. Likewise, we have also shown that Crkl phosphorylation was inhibited in a dose-dependent manner when exposed to N-(2-chloro-6-methylphenyl)-2-[[6-[4-(2-hydroxyethyl)-1-piperazinyl]-2-methyl-4-pyrimidinyl]amino]-5-thiazolecarboxamide (data not shown). Thus, such a Crkl assay will allows for an assessment of the enzymatic activity of BCR-ABL protein in a reproducible, quantitative fashion and be a useful means of assessing the ability of a combination therapy or more aggressive dosing regimen of the present invention to effectively overcome imatinib resistance, to inhibit BCR-ABL activity, or to inhibit BCR-ABL mutant activity.

Briefly, cells are lysed in 1% Triton X-100 buffer with protease and phosphatase inhibitors (see, e.g. A. Goga et al., Cell 82, 981 (1995)). Equal amounts of protein, as determined by the BioRad DC protein assay (Bio-RadLaboratories, Hercules, Calif.), are separated by SDS-PAGE, transferred to nitrocellulose and immunoblotted with phosphotyrosine antibody (4G10, Upstate Biotechnologies, Lake Placid, N.Y.), Abl antibody (pex5, (see, e.g. A. Goga et al., Cell 82, 981 (1995)), β-actin antibody (Sigma Chemicals, St. Louis, Mo.) or Crkl antiserum (Santa Cruz Biotechnology, Santa Cruz, Calif.). Immunoreactive bands are visualized by ECL (Amersham Pharmacia Biotech, Piscataway, N.J.). Several exposures are obtained to ensure linear range of signal intensity. Optimal exposures are quantified by densitometry using ImageQuant software (Molecular Dynamics, Sunnyvale, Calif.)).

### Example 5

### Methods for Examining Amplification of the BCR-ABL Gene in Mammalian Cells

An additional method of assessing the ability of a combination therapy or more aggressive dosing regimen of the present invention to effectively overcome imatinib resistance, to inhibit BCR-ABL activity, or to inhibit BCR-ABL mutant activity is provided. Specifically, dual-color fluorescence in situ hybridization (FISH) experiments can be performed to determine if BCR-ABL gene amplification is effectively diminished. The latter is based upon the appreciation in the art that BCR-ABL amplification is observed in imatinib-resistant and protein tyrosine kinase inhibitor resistant patients. Briefly, interphase and metaphase cells are prepared Grove, Ill.)). Cytogenetic and FISH characterization of metaphase spreads can be observed to assess if an inverted duplicate Ph-chromosome with interstitial amplification of the BCR-ABL fusion gene is present.

Alternatively, quantitative PCR analysis of genomic DNA obtained from patients can be used to assess if BCR-ABL gene amplification is present. Briefly, genomic DNA can be extracted from purified bone marrow or peripheral blood cells with the QiaAMP Blood Mini Kit (Qiagen, Inc., Valencia, Calif.). 10 ng of total genomic DNA is subjected to real-time PCR analysis with the iCycler iQ system (Bio-Rad Laboratories, Hercules, Calif.). A 361-bp cDNA fragment including ABL exon 3 is amplified using two primers (5′-GCAGAGTCAGAATCCTTCAG-3′ (SEQ ID NO: 8) and 5′-TTTGTAAAAGGCTGCCCGGC-3′ (SEQ ID NO: 9)) which are specific for intron sequences 5′ and 3′ of ABL exon 3, respectively. A 472-bp cDNA fragment of glyceraldehyde-3-phosphate dehydrogenase (GAPDH) is amplified using two primers (5′-TTCACCACCATGGAGAAGGC-3′ (SEQ ID NO: 13) and 5′-CAGGAAATGAGCTTGACAAA-3′ (SEQ ID NO: 14)) which are specific for sequences in exon 5 and exon 8 of GAPDH, respectively. Fold increase in ABL copy number can be determined by calculating the difference between threshold cycle numbers of ABL and GAPDH for each sample (DCt). A control can be used as a reference sample, DCt from each sample can be subtracted from DCt of control to determine D(DCt). Fold increase is then calculated as 2^{-D(DCt)}.

### Example 6

### Art Accepted Methods for Measuring Enzymological and Biological Properties of BCR-ABL Mutants

A variety of assays for measuring the enzymological properties of protein kinases such as Abl are known in the art, for example those described in Konopka et al., Mol Cell Biol. November 1985; 5(11):3116-23; Davis et al., Mol Cell Biol., January 1985; 5(1):204-13; and Konopka et al., Cell. Jul. 1, 1984; 37(3):1035-42 the contents of which are incorporated herein by reference. Using such assays the skilled artisan can measure the enzymological properties of mutant BCR-ABL protein kinases and to assess the ability of a combination therapy or more aggressive dosing regimen of the present invention to effectively overcome imatinib resistance, to inhibit BCR-ABL activity, or to inhibit BCR-ABL mutant activity.

A variety of bioassays for measuring the transforming activities of protein kinases such as Abl are known in the art, for example those described in Lugo et al., Science. 1995; 15(3):1286-93; Sitard et al., Blood. Mar. 15, 1994; 83(6):1575-85; Laneuville et al., Cancer Res. Mar. 1, 1994; 54(5):1360-6; Laneuville et al., Blood. Oct. 1, 1992; 80(7):1788-97; Mandanas et al., Leukemia. August 1992; 6(8):796-800; and Laneuville et al., Oncogene. February 1991; 6(2):275-82 the contents of which are incorporated herein by reference. Using such assays the skilled artisan can measure the phenotype of mutant BCR-Abl protein kinases.

Additional methods are disclosed in O'Hare et al. (Cancer Research, 65(11):4500-5 (2005), which is hereby incorporated by reference in its entirety.

### Example 7

### Dose Escalation Trial with Dasatinib

Acquired imatinib-resistance in patients with Ph-positive leukemia is frequently associated with somatic mutations in the BCR-ABL kinase domain. Dasatinib is more potent than imatinib for inhibiting BCR-ABL kinase activity and has preclinical efficacy against imatinib resistant mutations tested so far except for T3151. In a dose escalation trial of dasatinib, it was examined whether patients achieve significant reductions in the BCR-ABL level as measured by real-time quantitative PCR(RQ-PCR); the effect of dasatinib on mutant BCR-ABL clones in-vivo; and the contribution of mutations to relapse on dasatinib. Fourteen patients with accelerated phase or blast crisis CML, or Ph+ ALL (AP/BC) patients received a median of 5 months of dasatinib (1 to 11 months). Chronic phase (CP) CML patients (n=19) received a median of 12 months of dasatinib (3 to 21 months). Patients were tested 2 to 17 times by RQ-PCR (total 267 analyses) and direct sequencing (total 167 analyses). Prior to commencing dasatinib, 8 AP/BC and 15 CP patients had detectable mutations. For molecular analysis of these patients a>2-log reduction of BCR-ABL below the standardized baseline was considered significant. This level is equivalent to a complete cytogenetic response. A 3-log reduction defines a major molecular response (MMR), which is associated with a high progression free survival in imatinib treated patients. Six of 14 (43%) AP/BC and 7 of 19 (37%) CP patients achieved >2-log reductions (MMR in 4 (29%) and 4 (21%) patients respectively). Four of the 10 patients without detectable mutations at baseline were intolerant of imatinib. Overall, the incidence of a >2 log reduction was significantly higher in patients without a detectable mutation at baseline, P=0.026 (Table). The molecular response was maintained in 9 of the 13 patients (69%).

Mutations were detected at last analysis in all 23 patients with baseline mutations. The same mutation was present in 21 patients and 5 of these patients had an patients developed a mutation (all T315I). Of all patients with mutations that became detectable during dasatinib therapy (n=1 l), the T315I mutation became detectable in 7, including 1 CP patient. The detection of T315I was associated with significant rises in BCR-ABL of 2.5- to 185-fold in all bin 1 patient. Relapse was confirmed in 3 of these patients and 1 proceeded to transplant. Approximately 40% of all patients achieved significant molecular responses, which was maintained in the majority. The patients without detectable mutations prior to commencing dasatinib had a significantly better molecular response. Baseline mutations remained detectable in almost all patients including those with significant BCR-ABL reductions.

### Example 8

### Methods of Obtaining a Crystal Structure of Dasatinib Bound to Activated Abl Kinase Domain

Forward (CGGCCATATGCACGACCGAAAACCTGTATTTTCA GGGTGCCATGGATCCGTCCTCCCCCAACTACGACAAGTGGG) and reverse (GGCCGAATTCTTATTAGGTGCCACGTTTCCCCAGCTCCTTTTCCACTTCG) primers were used to amplify the kinase domain from a cDNA encoding human c-ABL (HSABL, accession X16416) and create an insert with a 5′-NdeI site, a sequence encoding a tobacco etch virus (TEV) protease cleavage site, the kinase domain, and a 3′-EcoRI site. This fragment was inserted into the baculovirus transfer vector pAcHLT-A (BD Biosciences PharMingen, San Jose, Calif.) cut with NdeI and EcoRI to construct the vector ABL/pAcHLT-A, encoding a NH₂-terminal (His)₆ fusion protein under control of the polyhedrin promoter. Following expression, purification, and removal of the (His)₆ fusion protein tag with TEV protease, the encoded protein contained non-native residues GAMDPS at the NH₂ terminal, human c-ABL S229-K512 (accession CAA34438), and mouse c-ABL residues R513-T515 at the COOH terminal. This protein was identical to 1FPU, except for the serine to asparigine difference between the mouse and human sequences, respectively, at amino acid residue 336 in the human c-ABL sequence.

ABL/pAcHLT-A was co-transfected with linearized baculovirus DNA (BaculoGold, BD Biosciences PharMingen) into Sf9 cells according to the manufacturer's protocol. A clonal population of recombinant virus was obtained by plaque purification. Recombinant virus was harvested and further amplified. Virus titer was determined using a standard plaque assay. After screening various variables, optimal conditions for (His)₆-ABL inoculated at a multiplicity of infection of five in the presence of 30% oxygen in the head space. Cells were harvested 72 hours after infection by centrifugation and frozen at −70° C. (His)₆-ABL kinase protein expression was estimated to be 5 mg/L of culture.

The ABL kinase domain was purified and combined with inhibitors as reported previously with minor modifications. Frozen cell paste from baculovirus-infected Sf9 cells was thawed and resuspended in 10 volumes of lysis buffer [50 mmol/L Tris-HCl (pH 8), 20 mmol/L NaCl, 10% (v/v) glycerol, 10 mmol/L DTT, Complete EDTA-free protease inhibitor cocktail (Roche, Indianapolis, Ind.)]. Cells were lysed by cavitation after nitrogen pressurization to 450 p.s.i. for 30 minutes at 4° C. The lysate was clarified by sedimentation at 100,000×gₘₐₓ for 40 minutes at 4° C. The supernatant was loaded onto a Q-Sepharose Fast Flow column equilibrated with 50 mmol/L Tris-HCl (pH 8), 20 mmol/L NaCl, 10% (v/v) glycerol, and 10 mmol/L DTT. The column was washed and eluted with a linear gradient of 20 to 500 mmol/L NaCl in 50 mmol/L Tris-HCl (pH 8), 10% (v/v) glycerol, and 15 mmol/L β-mercaptoethanol. (His)₆-ABL kinase was eluted in the gradient with 250 mmol/L NaCl and loaded onto a Ni-NTA Superflow (Qiagen, Valencia, Calif.) column equilibrated with 20 mmol/L imidazole. The column was eluted with a linear gradient of imidazole to 750 mmol/L. (His)₆-ABL kinase containing fractions were pooled and incubated at 50 μmol/L concentration at 4° C. for 18 hours with 1 unit recombinant TEV (rTEV) protease (Invitrogen, Carlsbad, Calif.) per 3 μg protein to remove the (His)₆ affinity tag. A 3× molar excess (150 μmol/L) of either imatinib or dasatinib was added from 15 mmol/L DMSO stocks to the completed rTEV digest. The ABL kinase inhibitor complex was purified and exchanged into the final buffer by chromatography on a Superdex 75 column in 20 mmol/L Tris-HCl (pH 8), 100 mmol/L NaCl, and 3 mmol/L DTT. The final yield of purified ABL kinase protein was ˜2.7 mg/L of culture. Purified ABL kinase inhibitor complexes were concentrated to 5 or 10 mg/mL with Amicon-ultra 10,000 molecular weight cut-off centrifugal ultrafilters (Millipore, Billerica, Mass.) before crystallization.

All crystallization trials were by hanging-drop, vapor-diffusion method at 4° C. Equal volumes of protein inhibitor and reservoir solutions were mixed to form 2 μL drops. Cocrystals of ABL-dasatinib and ABL-imatinib could be grown spontaneously based on reported conditions, but the initial crystals of neither complex produced useful diffraction. Attempts to improve crystal quality by using crushed ABL-dasatinib crystals as microseeds were unsuccessful. In contrast, larger, separated, and more regularly shaped crystals of the ABL-imatinib complex were produced by streak microseeding (using a cat whisker) crushed ABL-nucleate the growth of ABL-dasatinib crystals using the same procedure; diffraction quality crystals appeared in 3 days. The optimal reservoir solution was 22% (w/v) polyethylene glycol (PEG) 3350, 0.2 mol/L MgSO₄, and 0.1 mol/L MES (pH 6.5). Crystals were transferred by cryoloop into the cryoprotectant [30% (w/v) PEG 3350, 0.2 mol/L MgSO₄, and 0.1 mol/L MES (pH 6.5)] for 15 seconds and then relooped and flash cooled in liquid nitrogen.

Data collection was done at beamline X25 (National Synchrotron Light Source, Brookhaven National Laboratory, Upton, N.Y.), with the wavelength tuned to 1.1 Å and equipped with a nitrogen cryostream set to 100 K and an ADSC Quantum 315 detector positioned at 240 mm (ADSC, Poway, Calif.). Diffraction images were processed with Denzo and Scalepack from the HKL suite (HKL, Charlottesville, Va.;). Intensities were converted to structure factor amplitudes and placed on an absolute scale with Truncate from the CCP4 suite. Initial phases were calculated by molecular replacement by AMoRe, with the dimer from 1 FPU used as the search model. The structure was refined with CNX (Accelrys, Inc., San Diego, Calif.;) and modeled with Quanta (Accelrys).

Although both the ABL-imatinib and the ABL-dasatinib cocrystals were rod shaped, they grew as different crystal forms. The dasatinib cocrystals belonged to space group P4₃2₁2, with unit cell dimensions of a=105.2 Å and c=111.1 Å, and contained two molecules per asymmetric unit. This crystal form differs from those of previously reported ABL kinase crystal structures (PDB IDs 1M52, 1IEP, 1FPU, 1OPJ, 1OPK, and 1OPL). This change in crystal form was unexpected because cocrystals of the imatinib complex, which came from the same tray as those used as seeds, were shown to be of the same form as 1IEP and 1FPU (space group F222 with unit cell dimensions of a=111.9 Å, b=146.8 Å, and c=152.9 Å and contained two molecules per asymmetric unit). The structure was determined by molecular replacement with the 1FPU dimer used as the search model. A solution was not found when one monomer from 1FPU was used as the search model presumably because the noncrystallographic 2-fold rotation axis, which is nearly parallel with the X axis at approximately z=⅜ and y=½, is closely aligned with one of the crystallographic 2-fold screw axes. Instead, the dimer from 1 FPU was used. (Togarski et al., Cancer Res. (2006) 66:11 5790-5797, incorporated herein by reference in its entirety and for all purposes.)

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1. A method for determining the responsiveness of an individual with a BCR-ABL associated disorder to treatment with N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, comprising:
screening a biological sample from said individual for the presence of at least one mutation in a BCR-ABL polypeptide sequence; wherein the at least one mutation is a F317I mutation or a T315S mutation; and wherein the presence of the at least one mutation is indicative of the individual being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof, therapy.

2. The method of claim 1 wherein the at least one mutation is a F317I mutation.

3. The method of claim 2 wherein the sample is further screened for the presence of a T315A mutation.

4. The method of claim 1 wherein the at least one mutation is a T315A mutation.

5. The method of claim 1 wherein the individual has not previously been treated with a kinase inhibitor.

6. The method of claim 1 wherein the individual has been previously treated with a kinase inhibitor and has developed at least partial resistance to the kinase inhibitor.

7. The method of claim 6 wherein the kinase inhibitor is N-(2-chloro-6-methylphenyl)-2-[[6-[4-(2-hydroxyethyl)-1-piperazinyl]-2-methyl-4-pyrimidinyl]amino]-5-thiazolecarboxamide or a pharmaceutically acceptable salt, solvate, or hydrate thereof.

8. The method of claim 6 wherein the kinase inhibitor is imatinib, AMN107, PD180970, CGP76030, AP23464, SKI 606, or AZD0530.

10. The method of claim 9 wherein the leukemia is chronic myeloid leukemia (CML), Ph+ ALL, AML, imatinib-resistant CML, imatinib-intolerant CML, accelerated CML, or lymphoid blast phase CML.

11. The method of claim 1 wherein the sample is further screened for the presence of a E279K, F359C, F3591, L3641, L387M, F486S, D233H, T243S, M244V, G249D, G250E, G251S, Q252H, Y253F, Y253H, E255K, E255V, V256L, Y257F, Y257R, F259S, K262E, D263G, K264R, S265R, V268A, V270A, T272A, Y274C, Y274R, D276N, T277P, M278K, E279K, E282G, F283S, A288T, A288V, M290T, K291R, E292G, 1293T, P296S, L298M, L298P, V299L, Q300R, G303E, V304A, V304D, C305S, C305Y, T306A, F311L, I314V, T315I, E316G, F317L, M318T, Y320C, Y320H, G321E, D325H, Y326C, L327P, R328K, E329V, Q333L, A337V, V339G, L342E, M343V, M343T, A344T, A344V, I347V, A350T, M351T, E352A, E352K, E355G, K357E, N358D, N358S, F359V, F359C, F359I, I360K, I360T, L364H, L3641, E373K, N374D, K378R, V379I, A380T, A380V, D381G, F382L, L387M, M388L, T389S, T392A, T394A, A395G, H396K, H396R, A399G, P402T, T406A, S417Y, F486S or any combination thereof, mutation.

12. The method of claim 3 wherein the sample is further screened for the presence of a M244V, G250E, Q252H, Q252R, Y253F, Y253H, E255K, E255V, T315I, F317L, M351T, E355G, F359V, H396R, F486S, or any combination thereof, mutation.

13. The method of claim 3 wherein the sample is further screened for the presence of a M244V, E279K, F359C, F359I, L364I, L387M, or F486S, or any combination thereof, mutation.

14. The method of claim 3 wherein the sample is further screened for the presence of a L248R, Q252H, E255K, V299L, T315I, F317V, F317L, F317S, or any combination thereof, mutation.

15. A method of treating an individual suffering from a BCR-ABL-associated disorder comprising:
mutation or a T315A mutation, wherein the presence of the at least one mutation is indicative of the patient being at least partially resistant to N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, therapy; and
administering a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, solvate, or hydrate thereof, to the individual.

16. The method of claim 15 wherein the thiazolecarboxamide, or a pharmaceutically acceptable salt, solvate, or hydrate thereof, is administered at a higher dosage or dosing frequency if it is determined that the biological sample comprises a BCR-ABL polypeptide having the at least one mutation.

17. The method of claim 16, wherein the biological sample comprises a BCR-ABL polypeptide having the at least one mutation and the thiazolecarboxamide or pharmaceutically acceptable salt, hydrate, or solvate thereof is administered at a dosage of greater than 70 mg twice daily.

18. The method of claim 15 wherein the thiazolecarboxamide, or a pharmaceutically acceptable salt, solvate, or hydrate thereof, is administered in combination with a second therapy to treat the protein tyrosine kinase associated disorder in the individual.

19. The method of claim 18 wherein the second therapy is a tubulin stabilizing agent, a farnysyl transferase inhibitor, a BCR-ABL T315I inhibitor, a second protein tyrosine kinase inhibitor, or a combination thereof.

20. The method of claim 16 wherein the thiazolecarboxamide, or a pharmaceutically acceptable salt, solvate, or hydrate thereof, is administered in combination with a second therapy to treat the protein tyrosine kinase associated disorder in the individual.
inhibitor, or a combination thereof.

22. A method of identifying a compound that specifically binds to a BCR-ABL polypeptide, wherein the BCR-ABL polypeptide comprises at least one of a F317I mutation or a T315A mutation, comprising:
contacting a test compound with the BCR-ABL polypeptide; and
determining whether the BCR-ABL polypeptide specifically binds to the test compound.

23. The method of claim 22 further comprising a step of determining whether the test compound modulates the tyrosine kinase activity of the BCR-ABL polypeptide.

24. The method of claim 23 wherein the test compound inhibits the tyrosine kinase activity of the BCR-ABL polypeptide.

25. A method of determining whether a test compound modulates the tyrosine kinase activity of a BCR-ABL polypeptide, wherein the BCR-ABL polypeptide comprises at least one of a F317I mutation or a T315A mutation, comprising:
obtaining mammalian cells transfected with a construct encoding the BCR-ABL polypeptide so that the BCR-ABL polypeptide is expressed by the mammalian cells;
contacting the mammalian cells with the test compound; and
monitoring the mammalian cells for tyrosine kinase activity of the BCR-ABL polypeptide wherein a modulation in tyrosine kinase activity in the presence of the test compound identifies the test compound as a modulator of the BCR-ABL polypeptide.

26. The method of claim 25 wherein the test compound inhibits the tyrosine kinase activity of a BCR-ABL polypeptide.

27. A kit for use in determining treatment strategy for an individual with a BCR-ABL-associated disorder, comprising
a means for detecting a mutant BCR-ABL in a biological sample from said individual; and optionally
instructions for use and interpretation of the kit results.
6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate or solvate thereof.

29. The kit of claim 27 wherein said mutant BCR-ABL comprises a mutation at position 315 or 317.

30. The kit of claim 29 wherein said mutation at position 315 is selected from the group consisting of T315I and T315A.

31. The kit of claim 29 wherein said mutation at position 317 is selected from the group consisting of F317I, F317V, F317L, and F317S.

32. The kit of claim 29 further comprising a means for obtaining a biological sample from said individual.

33. A kit for use in treating an individual with a mutant BCR-ABL associated disorder, comprising:
a means for detecting a mutation at amino acid position 315 of a BCR-ABL from a biological sample from said individual;
a therapeutically effective amount of N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt or hydrate or solvate thereof; and
instructions for use of said kit.

34. The kit of claim 33 wherein said mutation at amino acid position 315 is selected from the group consisting of T315I and T315A.

35. A kit for use in treating an individual with a mutant BCR-ABL associated disorder, comprising:
a means for detecting a mutation at amino acid position 317 of a BCR-ABL from a biological sample from said individual;
pharmaceutically acceptable salt or hydrate or solvate thereof; and
instructions for use of said kit.

36. The kit of claim 35 wherein said mutation at amino acid position 315 is selected from the group consisting of F317I, F317V, F317L, and F317S.

37. A method of identifying amino acid positions within the BCR-ABL polypeptide that may confer at least partial resistance to a BCR-ABL inhibitor when said amino acid positions are mutated, comprising the steps of:
creating a co-crystal of the polypeptide with said BCR-ABL inhibitor; and
identifying the amino acid positions of said polypeptide that either contact, bond, interface or interact with said BCR-ABL inhibitor or that stabilize the contacting, bonding, interfacing, or interacting amino acids.

38. The method according to claim 37 wherein said BCR-ABL inhibitor is N-(2-chloro-6-methylphenyl)-2-[[6-[4-(2-hydroxyethyl)-1-piperazinyl]-2-methyl-4-pyrimidinyl]amino]-5-thiazolecarboxamide, or a pharmaceutically acceptable salt, hydrate, or solvate thereof.

39. The method according to claim 38 wherein said amino acid positions are selected from the group consisting of: 248, 299, 315, and 317.

40. The method according to claim 38 wherein said amino acid positions are selected from the group consisting of: 244, 248, 255, 290, 299, 313, 315, 316, 317, 318, 320, 321, and 380.
